SEQUENCE LISTING (1 sequence)

SEQ ID NO: 1 - protein, 134 aa, Homo sapiens
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

(19) United States
(12) Patent Application Publication
(10) Pub. No.: US 2004/0209810 A1
(43) Pub. Date: Oct. 21, 2004

(54) Method of treating Parkinson's disease in humans by intraputaminal infusion of glial cell-line derived neurotrophic factor

(76) Inventors: Steven S. Gill, Bristol (GB); Don M. Gash, Lexington, KY (US); Greg A. Gerhardt, Nicholasville, KY (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/784,547

(22) Filed: Feb. 23, 2004

Related U.S. Application Data
(60) Provisional application No. 60/449,789, filed on Feb. 24, 2003.

(51) Int. Cl. A61K 38/17

(52) U.S. Cl. 514/12

(57) ABSTRACT

A method of treating Parkinson's disease in humans is disclosed, wherein glial cell-line derive neurotrophic factor (GDNF) is chronically administered directly to one or both putamen of a human in need of treatment thereof. In one aspect of the present invention the GDNF is infused directly into one or both putamen through one or more indwelling intraparenchymal brain catheters connected to an implantable pump.

This application claims the benefit of U.S. Provisional Application No. 60,449,789, filed Feb. 24, 2003, which is hereby incorporated by reference in its entirety.

### FIELD OF THE INVENTION

The present invention relates generally to the field of neurobiology. More particularly, it concerns methods for treating Parkinson's disease in humans and related methods of restoring atrophic dopaminergic neurons and protecting dopaminergic neurons at risk of degeneration are also described.

### BACKGROUND OF THE INVENTION

Idiopathic Parkinson's disease (PD) is a neurodegenerative disorder characterized by the progressive death of selected populations of dopaminergic neurons, particularly within the pars compacta of the substantia nigra, with resulting reduction in striatal dopamine levels. There are approximately 500,000 specialized dopaminergic cells in the pars compacta of the substantia nigra of young adults. Symptoms of parkinsonism emerge when 75-80% of the dopaminergic innervation is destroyed. The consequential cardinal features, upon which clinical diagnosis is based, are tremor, rigidity, and akinesia/bradykinesia (Lang and Lozano, 1998). There are reportedly more than 1 million affected individuals in North America (Lang and Lozano, 1998), and an estimated overall prevalence within Europe of 1.6 per 100 population aged 65 years or older (de Rijk et al., 1997). Mortality among affected individuals is 2 to 5 times greater than among their age-matched, unaffected peers (Bennett et al., 1996; Morens et al., 1996; Louis et al., 1997), and life expectancy is markedly reduced (Morens et al., 1996). The single most consistent risk factor for the disease is age, and given the changing demography of industrialized nations, its burden upon their societies is likely to increase. Orally administered L-dopa, the immediate precursor of dopamine that is absorbed through the small intestine and is able, unlike dopamine itself, to cross the blood-brain barrier, remains the most effective treatment when combined with an aromatic amino acid decarboxylase inhibitor, currently widely available for Parkinson's disease (Koller, 2000; Jankovic, 2002). Although L-dopa does relieve the symptoms of PD (indeed, responsiveness to it, exhibited by more than 90% of patients, is one of the characteristic features of the disease [Lang and Lozano, 1998]), its use is not without problems. Its principal limitation, shared by dopamine agonists and more clearly apparent after several years of treatment, is the increasing inconsistency of patient responsiveness, manifested by motor fluctuations that take the form of distinct “wearing-off” and “on-off” phenomena (Nutt and Holford, 1996: Lang and Lozano, 1998; Koller, 2000; Jankovic, 2002). “Wearing-off,” also described as “end of dose deterioration,” is the term given to the relatively gradual and predictable decline in response to a dose of L-dopa that occurs over time, and this contrasts with “on-off” fluctuations in motor performance that are not clearly related to L-dopa dosing. In their early stages, motor fluctuations may be mitigated by approaches that prolong the actions of L-dopa (e.g., slow release formulations of the molecule or the co-administration of a catechol-O-methyltransferase inhibitor) or by the use of longer-acting synthetic dopamine agonists; however, these interventions cannot prevent an eventual increased unpredictability and lessened control of motor fluctuations and an increased incidence of dyskinesias during “on” periods (Lang and Lozano, 1998; Koller, 2000; Jankovic, 2002).

Neurotrophic factors are target-tissue-secreted molecules required for the development, guidance, and maintenance of innervating neurons. Specific retrograde transport to the neuronal soma is the hallmark of neuronal responsivity to a distinct neurotrophic factor (Oppenheim, 1989; 1991). Each neurotrophic factor affects the development and maintenance of specific populations of neurons, with some neurons responding to more than 1 neurotrophic factor.

Neurotrophic factors are expressed in different regions of the nervous system during different phases of development (Ernfors and Persson, 1991; Maisonpierre et al., 1990; Schecterson and Bothwell, 1992). Because of their specificities, neurotrophic factors have become attractive drug candidates for the treatment of neurodegenerative diseases that affect specific populations of neurons (Olson et al., 1992; Forander et al., 1996; Arenas et al., 1996).

Glial cell-line derived neurotrophic factor (GDNF) was first isolated from the culture medium of a rat glial cell line as a potent neurotrophic factor described as having relative specificity for dopaminergic neurons within dissociated rat embryonic midbrain cultures (Lin et al., 1993; Lin et al., 1994). After intracellular processing, GDNF is secreted as a glycosylated mature protein of 134 amino-acid residues. In its active form, GDNF is a disulfide-bonded homodimer of Mᵣ 32 kDa to 42 kDa (Lin et al., 1993; Lin et al., 1994). The human GDNF gene has been cloned, and recombinant human GDNF displaying full biologic activity has been expressed in E. coli (Lin et al., 1993).

Based on data collected in cell culture (Lin et al., 1993; Lin et al., 1994; Hou et al., 1996) and in rodent models of PD (Hoffer et al., 1994; Bowenkamp et al., 1995, Tomac et al., 1995a, Kearns et al., 1995), GDNF was thought to have significant therapeutic potential for the treatment of Parkinson's disease (PD) and amyotrophic lateral sclerosis (ALS)(Gash et al., 1996). However, important obstacles against the therapeutic application of GDNF to PD and other neurological disorders have been encountered. First, GDNF is a macromolecule that cannot pass through the blood-brain barrier, making it difficult to therapeutically deliver GDNF to the brain. Secondly, animal models are limited in their relevance to the human condition because of significant differences in the relative size of the brain. Intraventicular infusion of GDNF has, in fact, been attempted in PD patients but failed to result in therapeutic benefits. More specifically, 4 clinical studies in subjects with idiopathic PD (53 subjects; 50 of these were enrolled in the double-blind, placebo-controlled trial, and 38 of 50 received study drug) and 2 clinical studies in subjects with ALS (24 subjects; all of these were enrolled in the double-blind, placebo-controlled trial). In these studies, GDNF delivered to the cerebral ventricles (ICV) by monthly bolus dose (25 to 4000 μg per dose) or by chronic infusion (3 to 50 μg/day) failed to demonstrate clinical efficacy, i.e., no clinically or statistically significant improvements in signs or symptoms of PD or ALS were observed. Furthermore, almost all subjects (92% to 100%) experienced at least one adverse event during the studies. Mild-to-moderate nausea was the most frequently reported adverse event (approximately 70% to 90% incidence across all studies). Mild-to-moderate paresthesia was reported in 30% to 80% of subjects across all studies. Weight loss was reported in 14% to 63% of subjects across all studies. Serious adverse events were reported in 21% to 44% of subjects across all studies.

Another seemingly promising approach to treating PD that failed in clinical trials was the implantation of embryonic dopaminergic neurons into the brains of patients with PD. In a randomized, double-blind trial in which patients either received intraputaminal transplants of cultured embryonic mesencephalic tissue or were given sham surgery in which the dura mater was not penetrated, no clinical improvement was observed as a result of the transplants in patients over 60 years of age at one year after surgery, and only moderate improvement was apparent in those aged 60 years or less (Freed, C. et al., 2001). During continued follow-up of 12 to 36 months in patients who had received transplants, dystonia and dyskinesias had developed in a number of patients, all of whom had been <60 years of age at the time of surgery and each of whom had experienced clinical improvement during the first year after transplantation. Investigators in this study later reported findings that suggest that unbalanced increases in dopaminergic function resulted in the undesirable outcomes of neuronal transplantation for parkinsonism (Ma, Y. et al., 2002).

Renewed interest in ablative neurosurgical techniques has arisen as a consequence of increased understanding of the pathophysiology of basal ganglia and refinements in neurosurgical operating procedures, and pallidotomy and thalamotomy are again widely accepted options for consideration once a patient's condition has become increasingly difficult to manage using medication alone (Lang and Lozano, 1998; Jankovic, 2002). Regardless of the success or otherwise of such techniques to date, however, by their very nature, any side effects resulting from such interventions may very likely be irreversible. An alternative, less-irrevocable approach that simulates lesional effects, is deep-brain stimulation (DBS). In this procedure, electrical stimulation is provided on a long-term basis through implanted deep-brain electrodes and appears to result in improvements in motor function similar to those observed with ablative lesions. The mechanism by which this improvement occurs is not well understood but may involve the inhibition or disruption of neuronal activity (Lang and Lozano, 1998). However, DBS is neither neuroprotective nor neurorestorative and therefore does not halt the continual loss of remaining dopamine neurons or regenerate dopaminergic neurons already lost.

No satisfactory method exists to prevent or repair the damage caused by neuropathies, such as Parkinson's disease (parkinsonism) in human patients. Consequently, there continues to exist a long-felt need for safe and effective methods for the treatment and prevention of PD in humans. Ideally, such methods will stop the progression of the degenerative disease and even promote regeneration of the damaged neurons, without severe adverse side effects. Accordingly, it is an object of the present invention to provide such methods of treating PD in humans comprising the chronic, intraputaminal infusion of GDNF. This and other such objectives will be readily apparent to the skilled artisan from this disclosure.

### SUMMARY OF THE INVENTION

A first aspect of the present invention concerns a method of treating Parkinson's disease in a human comprising administering a pharmaceutical composition comprising a pharmaceutically effective dose of a glial cell line-derived neurotrophic factor (GDNF) protein product to one or both putamen of a human PD patient in need thereof. The GDNF protein product includes, without limitation, a pharmaceutically effective dose of r-metHuGDNF (a dimeric protein having an the amino acid sequence shown below in Table 1) or variants and/or derivatives thereof. The invention is based on the surprising discovery that continuous delivery of r-metHuGDNF to one or both putamen of a PD patient by means of an implantable pump and one or more indwelling catheters results in dramatic anti-parkinsonian and anti-dyskinetic effects which are further associated with impressive re-innervation and/or restoration of dopamine stores in previously dopamine deficient neurons in both non-human primate models of PD and human patients afflicted with PD. The methods of the present invention are contemplated to restore neural cell function in a patient having Parkinson's disease. Furthermore, the methods described herein are useful in repairing neural pathways damaged by Parkinson's disease in humans. Specifically, the methods described herein are capable of stimulating nerve regeneration, including re-innervation of damaged human brain tissue by dopaminergic neurons. In a preferred embodiment there is provided a method of increasing the function of dopaminergic neurons that comprises administering a pharmaceutically effective dose of r-metHuGDNF to one or both putamen of a human patient in need thereof.

Additionally provided are methods of treating cognitive disorders in humans that comprise administering a pharmaceutical composition comprising a pharmaceutically effective dose of a GDNF protein product to one or both putamen of a human in need thereof.

In still other embodiments of the present invention methods that comprise administering a pharmaceutical composition comprising a pharmaceutically effective dose of a GDNF protein product to one or both putamen of a human in need thereof may further comprise the step of assessing dopaminergic function in the brain of said human pre-operatively, and, optionally, assessing dopaminergic function in the brain of said human post-operatively at least once.

The methods of administering the GDNF to one or both putamen disclosed herein are contemplated as providing a prophylactic function in humans. Prophylactic administration may have the effect of preserving dopaminergic neural cell function in a human having, or at risk of having, Parkinson's disease. According to the invention, r-metHuGDNF administration to the human putamen is contemplated to preserve the integrity of the nigrostriatal pathway in the human brain. Accordingly, another embodiment of the present invention is a method of preventing degeneration of the nigrostriatal neuronal pathway or the loss of functional dopaminergic activity that comprises administering a pharmaceutical composition comprising a pharmaceutically effective dose of a GDNF protein product to one or both putamen of a human.

### BRIEF DESCRIPTION OF THE DRAWINGS

FIGS. 1(a)-(b) depict graphs of the behavioral response to daily infusions of r-metHuGDNF or vehicle. (a) Only the r-metHuGDNF recipients showed a significant and sustained behavioral improvement in their parkinsonian features of up to 3.5 points during the treatment period. (b) In the r-metHuGDNF recipients, consistent improvements of up to 60% were evident in bradykinesia, rigidity, balance and posture at peak effect. *P<0.05 vs baseline, same animals. LL, Lower limbs; UL, Upper limbs; T, Tremor.

FIGS. 2(a)-(i) depict graphs of the striatal levels of dopamine, HVA and DOPAC. As seen in the vehicle recipients, MPTP administration markedly reduced the levels of dopamine, HVA and DOPAC (a-i) in the medial (Med), intermediate (Int) and lateral (Lat) thirds of the right striatum (see j). In contrast, dopamine and DOPAC levels were significantly increased 233% and 180%, respectively, in the medial striatum on the lesioned right side of the r-metHuGDNF recipients (a, d). HVA levels were elevated 72%, 70% and 73% in the left medial, intermediate and lateral striatum, respectively (g-i). Values are expressed as ng/g wet weight of tissue. *P<0.05, r-metHuGDNF vs vehicle same side. Acb, accumbens; Cd, caudate nucleus; LV, lateral ventricle; Put, putamen.

FIGS. 3(a)-(j) illustrates the qualitative analysis of striatal dopamine fibers expressing TH. As seen in the low power (left panel) and high power (right panel) photomicrographs of vehicle recipients, the unilateral carotid artery infusion of MPTP virtually eliminated dopaminergic TH+ fibers in the right striatum (a, b, f, g). In comparison, chronic infusion of r-metHuGDNF stimulated TH+ fibers in the periventricular region of the right striatum (h, i, j). In one animal, the ventricular infusion of r-metHuGDNF greatly stimulated TH+ fibers in the caudate nucleus (arrows). The catheter tract is shown by an asterisk (*). The scale bars indicate 2 mm or 0.05 mm in distance. Cd, caudate; LV, ventricle; Put, putamen; R, right side.

FIG. 4 depicts a graph of the quantitative analysis of striatal dopamine fibers expressing TH. While a few residual TH+ fibers could be quantified in the right striatum of vehicle recipients, there was a significant five-fold increase in TH+ fibers in the periventricular striatal region of animals receiving r-metHuGDNF. TH+ fibers were most evident along the ventricular border of the right striatum, and gradually faded in a gradient from the ventricle to deeper into the parenchyma. One r-metHuGDNF recipient (#224s) was excluded from the analysis due to problems with sectioning of the tissue. *P<0.05, r-metHuGDNF vs vehicle right side.

FIG. 5(a) and 5(b) depicts patient assessments made using validated quality of life questionnaires: the 39-item Parkinson's Disease Questionnaire (PDQ 39; FIG. 5(a)) and the 36-item Medical Outcomes Study short form health survey (SF-36; FIG. 5(b)) before surgery and after 3, 6, 12, 18, and 24 months of chronic GDNF infusion.

FIG. 6(a) and 6(b) depicts the UPDRS scores for patients at 0, 3, 6, 12, 18, and 24 months following r-metHuGDNF infusion.

### DETAILED DESCRIPTION OF CERTAIN PREFERRED EMBODIMENTS

This invention is based on the discovery that continuous delivery of GDNF directly to one or both putamen by means of an implantable pump and at least one indwelling catheter in both non-human primate models of PD and human PD patients results in surprisingly dramatic anti-parkinsonian and anti-dyskinetic effects which are associated with impressive re-innervation and restoration of dopamine stores in previously dopamine deficient neurons.

In a primate model of PD, GDNF was administered by continuous infusion (both intraventricular and intraparenchymal) to achieve a magnitude of improvement in 3 cardinal features of PD—bradykinesia, rigidity, and postural stability—greater than that seen in studies using bolus administration. In a study in 13 female adult rhesus monkeys with neural deficits modeling the terminal stages of PD, the chronic infusion of 5 or 15 μg/day r-metHuGDNF into the lateral ventricle or the striatum, using programmable pumps, promoted restoration of the nigrostriatal dopaminergic system and significantly improved motor functions. The functional improvements were associated with pronounced upregulation and regeneration of nigral dopamine neurons and their processes innervating the striatum. In addition, chronic r-metHuGDNF treatment did not induce the side-effects generally associated with chronic administration of L-dopa. These findings are consistent with a regenerative effect of r-metHuGDNF on dopaminergic neurons in the nigrostriatal pathway, an effect that was not observed after monthly ICV bolus administration of r-metHuGDNF.

Furthermore, five patients with PD receiving r-metHuGDNF delivered directly into the putamen for two years showed a 57% improvement in the off-medication motor sub-score of the Unified Parkinson's Disease Rating Scale (UPDRS) and 63% improvement in the activities of daily living sub-score. Medication induced dyskinesias were reduced by 60% and were not seen off medication during chronic r-metHuGDNF delivery.

¹⁸F-dopa PET showed a significant 28% increase in dopamine storage in the putamen at 18 months suggesting a direct effect of r-metHuGDNF on dopamine function. Repeated measures analysis of variance (MANOVA) identified a significant difference in the ¹⁸F-dopa uptake constant (Ki) between the baseline scan and all post-operative scans in the posterior putamen (p<0.001). The increase was most marked at 24 months (60%, p<0.001).

Accordingly, a uniquely effective and long needed method of treating or preventing the pathological hallmarks of Parkinson's disease in humans as well as the devastating symptoms of PD is provided by the present invention.

The section headings used herein are for organizational purposes only and are not to be construed as limiting the subject matter described.

Abbreviations

In the preceding description and the experimental disclosure which follows, the following abbreviations apply:

6-OHDA 6-hydroxydopamine

ALS amyotrophic lateral sclerosis

ASA acute systemic anaphylaxis

AUC area under the concentration vs time curve

CAPIT Core Assessment Program for Intracerebral Transplantations.

CAPS 3-(cyclohexylamino)-1-propanesulfonic acid

CHO Chinese hamster ovary

CI continuous infusion

CSF cerebrospinal fluid

CT computed tomography

DA dopamine, dopaminergic

DOPAC 3,4-dihydroxyphenylacetic acid

E. coli Escherichia coli

FCA Freund's Complete Adjuvant

GDNF glial cell line-derived neurotrophic factor

GLP Good Laboratory Practice

HPLC high-performance liquid chromatography

HVA homovanillic acid

Icv intracerebroventricular

IM intramuscular

ISN intranigral

IT intrathecal

IV intravenous

L-dopa 3,4-dihydroxyphenylalanine (levodopa)

r-metHuGDNF recombinant-methionyl human GDNF

MPTP 1-methyl-4-phenyl-1,2,3,6-tetrahydropyridine

PD Parkinson's disease

PET positron-emission tomography

pmn progressive motor neuropathy

Ret receptor tyrosine kinase

r-metHuGDNF recombinant-methionyl human GDNF

SC subcutaneous

SDS-PAGE sodium dodecylsulfate-polyacrylamide gel electrophoresis

SEM standard error of the mean

TGF transforming growth factor

TH tyrosine hydroxylase

TH+ tyrosine hydroxylase positive

UPDRS Unified Parkinson's Disease Rating Scale

Definitions

Unless otherwise noted, technical terms are used according to conventional usage. As utilized in accordance with the present disclosure, the following terms shall be understood to have the following meanings:

As used herein, the term “catheter” refers to any tubular medical device for insertion into a cavity, tissue, organ, or any substructure thereof of a living mammal to permit injection of a therapeutic agent. As particularly used here, a catheter is used to deliver r-metHuGDNF to the brain or substructures thereof such as the putamen. An “indwelling” catheter is one that is implanted and left in place for protracted periods, such as fifteen minutes or longer.

As used herein, the phrase “catheter system” refers to the combination of at least one catheter and at least one accessory device including, but not limited to, an anchor, stylet, guide tube, guide wire or a combination thereof.

“Continuous delivery” or “chronic infusion” are interchangeable and are intended to mean delivery of a substance over a period of time such that the procedure is distinguished from “bolus” delivery. Continuous delivery generally involves the delivery of a substance over a period of time without interruption. The rate of delivery need not be constant, and the period of delivery need not be very long, i.e., the period of constant delivery may be over a period of maybe half an hour or an hour or a few hours, but may also be over a period of days, weeks, months, or even years.

“Admixing” as used herein denotes the addition of an excipient to a polypeptide of interest, such as by mixing of dry reagents or mixing of a dry reagent with a reagent in solution or suspension, or mixing of aqueous formulations of reagents.

“Excipient” as used herein denotes a non-therapeutic agent added to a pharmaceutical composition to provide a desired consistency or stabilizing effect.

“Implanted” means placed within the body, and maintained at that location for some extended period of time. As used herein it is intended that the period of time during which the implanted object is maintained in place will be, in general, considerably greater than that customarily required to introduce a bolus of a substance, such as a drug. For example, a catheter used in a method of the invention may be placed within a tissue or organ such that the catheter so implanted is intended to remain at the site of implantation for some extended period of time. Some of the drug delivery apparatuses that may be used in the methods of the invention, for example the drug pumps and/or catheters, are designed to be implanted for periods greater than a month and even years and to deliver drug during this period. A drug delivery apparatus may be implanted, for example, subcutaneously, or within a tissue or organ, or within a body cavity such as the peritoneal cavity, infraclavicular space, the thoracic cavity, the pelvic cavity, or any other cavity or location that is convenient for delivery of the intended substance. A catheter may be implanted into a tissue, for example into brain tissue, and may be affixed in place by fixing the catheter to another tissue, such as bone, e.g., the skull, or cartilage, using an adhesive or screws, clamps, sutures or any other suitable fixing means.

The phrases “dopaminergic dysfunction”, “dopaminergic dysregulation”, “dopaminergic degeneration”, “dopamine depleted”, “dopamine deficient”, or grammatical equivalents thereof, may be used interchangeably herein. All such phrases are intended to encompass at least one of the following conditions or disorders: Parkinson's disease, neuronal dopamine deficit, dopaminergic neuron deficit, dopaminergic neuron lesions, hypo-dopaminergic innervation, dopamine synthesis incapacity, dopamine storage incapacity, dopamine transport incapacity, or dopamine uptake incapacity. Dopaminergic dysfunction can be evidenced by analyzing factors including, but not limited to, the following: 1) the number of TH expressing neurons 2) size of dopamine neuronal cells 3) dopamine metabolite levels 4) dopamine uptake, 5) dopamine transport, 6) neuronal dopamine uptake, 7) dopamine transporter binding, 8) quantal size of terminal dopamine release, 9) rate of dopamine turnover, 10) TH+ cell count, 11) TH+ innervation density and 12) TH+ fiber density.

The phrase “target site”, or a grammatical version thereof, refers to the site for intended delivery of a substance, such as a drug. In particular embodiments of the present invention, a preferred target site is an area of dopaminergic degeneration or dopaminergic dysfunction within the brain of a human afflicted with Parkinson's disease. More preferably, the target site is the central area of the putamen. Even more preferably, the target site is the posterior area of the putamen. Most preferably, the target site is the postero-dorsal area of the putamen. Furthermore, a particular target site may be targeted unilaterally or bilaterally with respect to the hemispheres of the brain.

“Proximal end” is a relative term, and generally refers to the end of a device, such as a catheter that is nearest to the operator (i.e., the surgeon) and is furthest away from the treatment site. In the present invention a catheter has a proximal end that may be communicably attached to an access port or drug delivery apparatus, such as a pump, or reservoir.

“Tyrosine hydroxylase-positive” or “TH+” is intended to refer to the presence of tyrosine hydroxylase in a referenced nervous tissue as indicated by the results from any technique known in the art as a means to detect and/or measure tyrosine hydroxylase, tyrosine hydroxylase encoding mRNA, or tyrosine hydroxylase activity.

“Distal end” is a relative term and generally refers to the end of a device, such as a catheter, that is furthest away from the operator (i.e., the surgeon) and is closest to the treatment site. In the present invention the distal end of a catheter may be communicably attached to an opening that allows for the delivery of drug to the target site.

“Drug delivery apparatus” as used herein includes but is not limited to, a drug reservoir and/or a drug pump of any kind, for example an osmotic pump, an electromechanical pump, an electroosmotic pump, an effervescent pump, a hydraulic pump, a piezoelectric pump, an elastomeric pump, a vapor pressure pump, or an electrolytic pump. Preferably, such a pump is implanted within the body.

Throughout this specification, reference to the term “GDNF” or the phrase “GDNF protein product” or “GDNF polypeptide”, all of which are used interchangeably, refers to glial cell line-derived neurotrophic factor from any species, including murine, bovine, ovine, porcine, equine, avian, and preferably human, in native sequence or in genetically engineered variant form, including, without limitation, biologically active fragments, analogs, variants, (including, insertion, substitution, and deletion variants) and derivatives thereof, and from any source, whether natural, synthetic, or recombinantly produced.

Exemplary GDNF polypeptides useful in the present invention include, without limitation, any of GDNF protein products described in U.S. Pat. Nos. 5,731,284, 6,362,319, 6,093,802, and 6,184,200 (all of which are hereby incorporated by reference in their entireties). Preferred GDNF protein products for use in the methods of the present invention include, but are not limited to, r-metHuGDNF, a recombinant GDNF protein produced in E coli which has an amino acid sequence identical to native mature human GDNF with the addition of an amino terminal methionine. Thus, r-metHuGDNF consists of 135 amino acids. Seven of the amino acids are cysteines, which are involved in one intermolecular disulfide bond and three intramolecular disulfide bonds. In its active form, r-metHuGDNF is a disulfide-bonded homodimer. The primary amino acid sequence of monomeric r-metHuGDNF is provided in Table 1.

TABLE 1
Primary Amino Acid Sequence of r-metHuGDNF

Amino
Acid

| | |
|---|---|
| Primary Amino Acid Sequence | No. |

| | | | | | | | | | | | |
|---|---|---|---|---|---|---|---|---|---|---|---|
| H₂N-Met | Ser | Pro | Asp | Lys | Gln | Met | Ala | Val | Leu | Pro | 10 |
| | Arg | Arg | Glu | Arg | Asn | Arg | Gln | Ala | Ala | Ala | 20 |
| | Ala | Asn | Pro | Glu | Asn | Ser | Arg | Gly | Lys | Gly | 30 |
| | Arg | Arg | Gly | Gln | Arg | Gly | Lys | Asn | Arg | Gly | 40 |
| | Cys | Val | Leu | Thr | Ala | Ile | His | Leu | Asn | Val | 50 |
| | Thr | Asp | Leu | Gly | Leu | Gly | Tyr | Glu | Thr | Lys | 60 |
| | Glu | Glu | Leu | Ile | Phe | Arg | Tyr | Cys | Ser | Gly | 70 |
| | Ser | Cys | Asp | Ala | Ala | Glu | Thr | Thr | Tyr | Asp | 80 |
| | Lys | Ile | Leu | Lys | Asn | Leu | Ser | Arg | Asn | Arg | 90 |
| | Arg | Leu | Val | Ser | Asp | Lys | Val | Gly | Gln | Ala | 100 |
| | Cys | Cys | Arg | Pro | Ile | Ala | Phe | Asp | Asp | Asp | 110 |
| | Leu | Ser | Phe | Leu | Asp | Asp | Asn | Leu | Val | Tyr | 120 |
| | His | Ile | Leu | Arg | Lys | His | Ser | Ala | Lys | Arg | 130 |
| | Cys | Gly | Cys | Ile | COOH | | | | | | 134 |

The GDNF protein products useful in the present invention may be isolated or generated by any means known to those skilled in the art. Preferably, GDNF is recombinantly produced. In a preferred method, the GDNF is cloned and its DNA expressed, e.g., in mammalian cells or bacterial cells. Exemplary methods for producing GDNF protein products useful in the present invention are described in U.S. Pat. Nos. 6,362,319, 6,093,802 and 6,184,200 (all of which are hereby incorporated by reference in their entireties).

GDNF pharmaceutical compositions typically comprise a therapeutically effective amount of at least one GDNF protein product and one or more pharmaceutically and physiologically acceptable formulation agents. Suitable formulation agents include, but are not limited to, antioxidants, preservatives, coloring, flavoring and diluting agents, emulsifying agents, suspending agents, solvents, fillers, bulking agents, buffers, vehicles, diluents, excipients and/or pharmaceutical adjuvants. For example, a suitable vehicle may be, physiological saline solution, citrate buffered saline, or artificial CSF, possibly supplemented with other materials common in compositions for parenteral administration. Neutral buffered saline or saline mixed with serum albumin are further exemplary vehicles. Those skilled in the art would readily recognize a variety of buffers that could be used in the compositions, and dosage forms used in the invention. Typical buffers include, but are not limited to pharmaceutically acceptable weak acids, weak bases, or mixtures thereof. Preferably, the buffer components are water soluble materials such as phosphoric acid, tartaric acids, lactic acid, succinic acid, citric acid, acetic acid, ascorbic acid, aspartic acid, glutamic acid, and salts thereof.

The primary solvent in a vehicle may be either aqueous or non-aqueous in nature. In addition, the vehicle may contain other pharmaceutically-acceptable excipients for modifying or maintaining the pH, osmolarity, viscosity, clarity, color, sterility, stability, rate of dissolution, or odor of the formulation. A preferred pharmaceutical composition of GDNF comprises a therapeutically effective amount of at least one GDNF protein and a pharmaceutically acceptable vehicle. More preferably, the pharmaceutically acceptable vehicle is an aqueous buffer. More preferably, the vehicle comprises sodium chloride at a concentration of about 100 mM to about 200 mM and sodium citrate at a concentration of about 5 mM to about 20 mM. Even more preferably, the vehicle comprises sodium chloride at a concentration of about 125 mM to about 175 mM and sodium citrate at a concentration of about 7.5 mM to about 15 mM. Even more preferably, the vehicle comprises sodium chloride and sodium citrate at a concentration of about 150 mM and about 10 mM, respectively. Most preferably, the GDNF pharmaceutical composition is formulated as a liquid with 10 mM sodium citrate and 150 mM sodium chloride at a pH of 5.0.

The pharmaceutical composition may contain still other pharmaceutically-acceptable formulation agents for modifying or maintaining the rate of release of GDNF protein product. Such formulation agents are those substances known to artisans skilled in formulating sustained release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712 (the disclosure of which is hereby incorporated by reference).

Once the therapeutic composition has been formulated, it may be stored in sterile vials as a solution, suspension, gel, emulsion, solid, or dehydrated or lyophilized powder. Such formulations may be stored either in a ready to use form, a lyophilized form requiring reconstitution prior to use, or a liquid form requiring dilution prior to use. Preferably, the GDNF pharmaceutical composition is provided in sterile single-use vials at a concentration of 10 mg/mL and stored frozen at a temperature of −2-8° C. until use. Immediately prior to administration, the GDNF protein product should be thawed and appropriately diluted with a sterile citrate buffered saline (pH 5.0) consisting of 150 mM sodium chloride and 10 mM sodium citrate.

In the methods of the present invention, GDNF is chronically administered to a site of dopaminergic dysfunction in the human brain by means of an implantable pump and one or more catheters. Preferably, the region of a PD patient's brain targeted for chronic delivery of GDNF is determined by assessing biomarkers of PD disease or disease progression including, but not limited to, the number of TH expressing neurons 2) size of dopamine neuronal cells 3) dopamine metabolite levels 4) dopamine storage, 5) dopamine transport, 6) neuronal dopamine uptake, 7) dopamine transporter binding, 8) quantal size of terminal dopamine release, 9) rate of dopamine turnover, 10) TH+ cell count, 11) TH+ innervation density and 12) TH+ fiber density. Even more preferably, GDNF is chronically infused directly into a region of the human brain which is severely dopamine depleted. Even more preferably, the region of a PD patients brain which is severely dopamine depleted and, therefore, targeted for chronic delivery of GDNF is determined by neuroimagery of the brain, or regions thereof. Even more preferably, the neuroimagery technique used to determine the site of chronic infusion of GDNF is selected from the group consisting of ¹⁸F-fluorodopa positron emission tomography (¹⁸F-dopa PET)¹³ and ¹²³I-2β-carboxymethoxy-3β-(4-iodophenyl)tropane uptake on single-photon emission tomography (¹²³I-β-CIT SPECT). In an even more preferred embodiment of the present invention, GDNF is chronically infused directly into at least one dopaminergic dysfunctional putamen of a PD patient. Even more preferably, GDNF is chronically infused directly into the posterior region of at least one dopaminergic dysfunctional putamen of a PD patient. Most preferably, GDNF is chronically infused directly into at least one dopaminergic dysfunctional postero-dorsal putamen of a PD patient.

A number of drug delivery apparatus, catheters, catheter systems and combinations thereof have been developed for the dispensing of medical substances to specific sites within the body and are all readily available to those skilled in the art for use in the methods of the present invention. Therefore, one may use prior art drug delivery devices, catheters, and catheter systems for delivering the GDNF compositions to the target site of the brain of the patient at specified concentrations and/or at specified times and/or at different delivery rates. By way of illustration and not limitation, in the methods of the present invention one may use the technology described in U.S. Patent Publication No. US20030120262, US20030208184, or US20030225372 or U.S. Pat. Nos. 4,931,050, 4,838,887, 5,207,666, 4,714,462, 5176,641; 3,923,060, 4,003,379, 4,588,394, 4,447,224, 5,575,770, 4,978,338, 5,908,414, 5,643,207, 6,589,205 or 6,592,571. The entire disclosure of each of these U.S. Patent Applications and U.S. Patents is hereby incorporated by reference into this specification. A preferred drug delivery apparatus useful in the context of the present invention includes one described in U.S. Pat. No. 5,752,930 or U.S. Patent Application No. US20030216714 (which are hereby incorporated by reference in their entireties). A more preferred drug delivery apparatus useful in the context of the present invention includes one described in U.S. Pat. No. 6,620,151 (which is hereby incorporated by reference in its entirety). An even more preferred drug delivery apparatus useful in the context of the present invention includes one described in U.S. Patent Application No. US20030216714 (which is hereby incorporated by reference in its entirety). Most preferably the drug delivery apparatus used in the context of the present invention is one described in U.S. Pat. Nos. 4,146,029, 4,013,074, or 4,692,147, (which are hereby incorporated by reference in their entireties) commercial embodiments thereof including, but not limited to, the Synchromed® I, Synchromed® EL, and Synchromed® II infusion pumps (Medtronic, Inc., Minneapolis, Minn.).

In another embodiment of the present invention, in conjunction with any of the above or below embodiments, a number of catheters and catheter systems have been developed for the dispensing of agents, such as drugs, to specific sites in the body and are readily available to those skilled in the art for use in the methods of the present invention. By way of illustration and not limitation, in the methods of the present invention one may use the technology described in U.S. Patent Publication No. 20030216700, 20030199831, or 20030199829 or U.S. Pat. No. 6,319,241. The entire disclosure of these U.S. Patent Applications and the United States patent is hereby incorporated by reference into this specification. A preferred catheter or catheter system useful in the context of the present invention includes, but is not limited to, an intraparenchymal infusion catheter or catheter system described in International Patent Application Publication No: WO 02/07810 or WO03/002170 or U.S. Pat. Nos. 5,720,720, 6,551,290 or 6,609,020.

The entire disclosure of each of these Patent Applications and United States patents is hereby incorporated by reference into this specification. An even more preferred catheter or catheter system useful in the context of the present invention includes, but is not limited to, an intraparenchymal infusion catheter or catheter system described in International Patent Application Publication No: WO 03/077785 (which is hereby incorporated by reference in its entirety). A most preferred catheter or catheter system useful in the context of the present invention is an intraparenchymal infusion catheter or catheter system described in U.S. Pat. No. 6,093,180 (which is hereby incorporated by reference in its entirety).

In another embodiment of the methods of the present invention, in conjunction with any of the above or below embodiments, a therapeutically effective dose of GDNF is chronically infused directly into one or both putamen of a human PD patient. The phrase “therapeutically effective dose” or “pharmaceutically effective dose”, which are used interchangeably herein, refers to that amount of GDNF sufficient to result in any amelioration, impediment, prevention or alteration of any biological symptom generally associated with a neurodegenerative disorder including, without limitation, PD by one skilled in the relevant art. In a preferred embodiment of the present invention, in conjunction with any of the above or below embodiments, GDNF is chronically infused directly into a human putamen at a dose of about 1 μg/putamen/day to about 100 μg/putamen/day. More preferably, GDNF is chronically infused directly into a human putamen at a dose of about 5 μg/putamen/day to about 50 μg/putamen/day. Even more preferably, GDNF is chronically infused directly into a human putamen at a dose of about 10 μg/putamen/day to about 75 μg/putamen/day. Even more preferably, GDNF is chronically infused directly into a human putamen at a dose of about 15 μg/putamen/day to about 50 μg/putamen/day. Even more preferably, r-metHuGDNF is chronically infused directly into human putamen at a dose of about 20 μg/putamen/day to about 40 μg/putamen/day. Even more preferably, r-metHuGDNF is chronically infused directly into a human putamen at a dose of about 25 μg/putamen/day to about 30 μg/putamen/day. Even more preferably, r-metHuGDNF is chronically infused directly into a human putamen at a dose of about 15 μg/putamen/day to about 30 μg/putamen/day. Most preferably, r-metHuGDNF is chronically infused directly into a human putamen at a dose of about 25 μg/putamen/day to about 30 μg/putamen/day.

The inventive method has the effect, upon application to parkinsonian patients, of significantly reducing symptoms of Parkinson's disease, the resulting improved condition of the patient continuing for at least 30 months. In particular, a clear improvement of disease-specific symptoms was obtained with the inventive method insofar as motoricity, fine motoricity, and fine dexterity. In addition, mobility and concentration power was increased and reaction time was decreased. Pronunciation, facial expressiveness, posture, sense of smell, libido, sexual function, and emotional condition were improved and state of mind was brightened.

In yet another embodiment of the present invention, GDNF can be used as a cognitive enhancer, to enhance learning, particularly as a result of dementias or trauma, or to inhibit cognitive decline and/or dementia, for example, in patients with PD. Alzheimer's disease, which has been identified by the National Institutes of Aging as accounting for more than 50% of dementia in the elderly, is also the fourth or fifth leading cause of death in Americans over 65 years of age. Four million Americans, 40% of Americans over age 85 (the fastest growing segment of the U.S. population), have Alzheimer's disease. Twenty-five percent of all patients with Parkinson's disease also suffer from Alzheimer's disease-like dementia. Applicants have shown here for the first time that chronic intraputaminal administration of GDNF has application in treating or preventing cognitive disorders in humans. In particular, intraputaminal administration of GDNF has application in treating or preventing cognitive disorders and/or Alzheimer's disease-like dementia associated with PD.

The present invention is also directed to kits which comprise:

(a) one or more supplies of a pharmaceutical composition comprising a GDNF protein product and a pharmaceutically acceptable vehicle, excipient, or diluent; and

(b) supplies adapted for refilling an implanted drug delivery device with said composition.

In a preferred embodiment of the kits the GDNF protein product is r-metHuGDNF. In another preferred embodiment, the kit further comprises at least one syringe. In another preferred embodiment, the kit further comprises; and one or more supplies of a pharmaceutically acceptable diluent. More preferably, the pharmaceutically acceptable diluent is citrate buffered saline consisting of about 150 mM sodium chloride and about 10 mM sodium citrate, pH of about 4.5 to about 5.5. Even more preferably, the pharmaceutically acceptable diluent is citrate buffered saline consisting of 150 mM sodium chloride and 10 mM sodium citrate, pH 5.0. Even more preferably, the kit further comprises instructions for diluting the pharmaceutical composition with the diluent. Even more preferably, the kit further comprises instructions for refilling said drug delivery device.

In certain examples, the kit comprises multiple sealed containers, including, but not limited to, removable sealed containers that contain the pharmaceutical composition, diluent, or supplies provided for refilling an implanted drug delivery device with said composition. Several containers may contain the same provision. Furthermore, a container may contain more than one provision.

Preferably, the pharmaceutical compositions, diluents, and supplies provided for refilling an implanted drug delivery device with the GDNF pharmaceutical composition are provided sterile in sealed containers in the kit.

In some embodiments of the kit, the pharmaceutical composition is provided in powder or other dry form. The powder or other dry form may be combined with a liquid, including, without limitation, the diluent for purposes of reconstituting the pharmaceutical composition in a liquid form for use in refilling the implanted drug delivery device.

### EXAMPLES

The following examples, including the experiments conducted and results achieved are provided for illustrative purposes only and are not to be construed as limiting the invention.

### Example 1

### Treatment of Advanced Parksonian like Neural Deficits in Non-Human Primates with Chronic, Controlled r-metHuGDNF Infusion into the Brain

To assess the restorative actions of r-metHuGDNF under conditions where neuroprotection would have only a minor role, the late stages of human PD were modeled using rhesus monkeys with stable, advanced hemi-parkinsonian features induced by the neurotoxin 1-methyl-4-phenyl-1,2,3,6-tetrahydropyridine (MPTP) (Bankiewicz et al., 1983; Smith et al., 1993; Emborg-Knott and Domino, 1998). In this model, MPTP infusion through the right carotid artery results in an approximate 75% loss of dopamine neurons expressing the phenotypic marker tyrosine hydroxylase (TH) in the right substantia nigra and a greater than 99% depletion of dopamine in the right putamen (Gash et al., 1996). These reductions are comparable with advanced human parkinsonism where cell counts typically show a 60-70% loss of nigral dopamine neurons (Jellinger, 1986) and a 99% dopamine depletion in the putamen (Kish et al., 1988). Subcutaneously implanted programmable pumps connected to catheters implanted into either the right lateral ventricle adjacent to the striatum or bilaterally into the striatum were used to continuously deliver controlled doses of r-metHuGDNF or vehicle to the MPTP-injured nigrostriatal system. Behavioral recovery was quantified using standardized videotaped tests; regeneration of the nigrostriatal dopamine system was analyzed by quantitative morphology and high performance liquid chromatography (HPLC) measurements of dopamine levels. The levels of r-metHuGDNF promoting regeneration of the nigrostriatal system and motoric recovery were quantified by enzyme-linked immunosorbent assay (ELISA) and HPLC.

Animal Procedures

All procedures were conducted in the Laboratory Animal Facilities of the University of Kentucky, which are fully accredited by the Association for Assessment and Accreditation of Laboratory Animal Care International (AAALACI). Veterinarians skilled in the health care and maintenance of nonhuman primates supervised all animal care. The University of Kentucky's Animal Use Committee approved all protocols.

Thirteen adult (13±0.6 years old) female rhesus monkeys (Macaca mulatta) received right intracarotid artery infusions of 0.4 mg/kg MPTP to induce continuously expressed parkinsonian features (Ovadia et al., 1995). The animals were monitored for a minimum of two months to ensure that the parkinsonian features expressed were stable. At this point, using stereotaxic procedures guided by magnetic resonance imaging, a catheter (1 mm O.D., Medtronic Inc., Minneapolis, Minn.) was surgically implanted into the right lateral ventricle adjacent to the striatum (n=8) or bilaterally into the central part of the putamen (n=5). The catheter(s) were then connected via a flexible polyurethane tubing to a programmable pump (SynchroMed™ model 8616-10; Medtronic Inc., Minneapolis, Minn.) subcutaneously implanted in the lateral abdominal region (Grondin et al., 2001). The catheters were implanted bilaterally into the putamen in order to parallel potential bilateral effects of the ventricular delivery on nigral neurons (Gash et al., 1996). Placement of the catheter(s) was verified by magnetic resonance imaging. The animals were anesthetized with isoflurane (1-3%) during these procedures. The vehicle (10 mM citrate, 150 mM NaCl buffer) was infused daily in all thirteen animals during the first month following the surgery and continued in five control animals for an additional three months (n=3 intraventricular, n=2 intraputamenal). The remaining eight animals received infusions of nominally 7.5 μg r-metHuGDNF (Amgen, Thousand Oaks, Calif.) per day over the same three month period (n=5 intraventricular, n=3 intraputamenal). Four of the animals responded to this dose. In order to achieve a similar level of behavioral improvement among animals (>2 points on the rating scale), the daily dose was increased to nominally 22.5 μg in the other four animals (n=2 intraventricular, n=2 intraputamenal). The pumps were refilled with r-metHuGDNF or vehicle every 4 weeks by injections through the skin into a fill port (Grondin et al., 2001). To estimate actual dose levels of r-metHuGDNF chronically infused into the brain, residual r-metHuGDNF F solutions were removed from the pumps of three animals at the four, eight and twelve week time points of r-metHuGDNF infusion. Protein loss from adsorption to the pump was estimated by an ELISA essay (Amgen, Thousand Oaks, Calif.), while the stability of r-metHuGDNF after 4 weeks in the pumps at 37° C. (body temperature) was determined by reverse phase HPLC.

Motor functions were assessed using previously published nonhuman primate parkinsonian scale, patterned after the human Unified Parkinson's Disease Rating Scale (Ovadia et al., 1995). Two hours of weekly standardized tests were conducted pre- and post-treatment, and were evaluated from coded videotapes (Ovadia et al., 1995). A review of this widely used approach is published elsewhere (Imbert et al., 2000). In addition, the tapes were analyzed to determine if the animals displayed side effects from the treatment (Miyoshi et al., 1997). Post MPTP/baseline scores were defined as the averaged scores of two videotaping sessions conducted prior to implanting the pumps. Because the rating scale is non-linear, the cumulative scores obtained weekly in the control and the r-metHuGDNF recipients were analyzed using a non-parametric Friedman test for related samples followed with a post hoc analysis by a non-parametric Wilcoxon signed rank test on pairs of related samples. For a more in depth analysis of the behavioral response to r-metHuGDNF, the rating scale was broken into its different components, namely, posture, balance, rigidity, tremor and bradykinesia. For each rated category, the post MPTP/baseline scores and the scores obtained at peak effect in the treated group, were compared using a Wilcoxon signed rank test on pairs of related samples. Each animal was used as its own control.

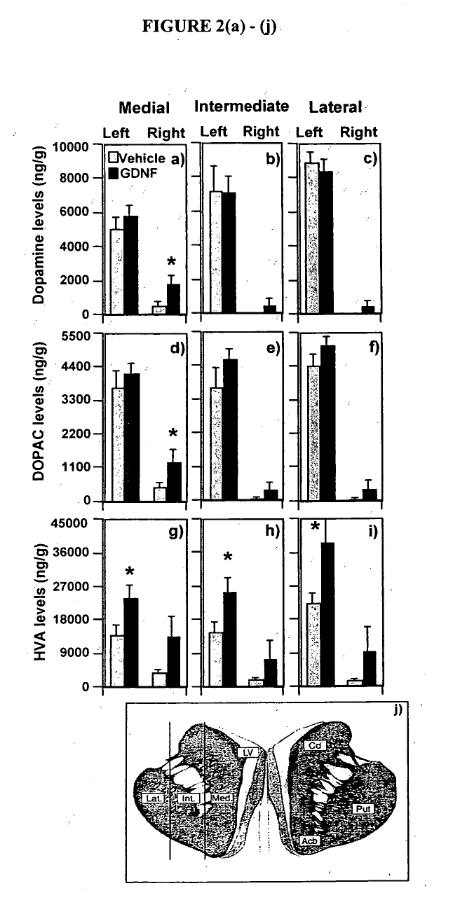

After receiving an initial dose of ketamine hydrochloride (20-25 mg/kg), the animals were deeply anesthetized using pentobarbital sodium (20 mg/kg) and transcardially perfused with heparinized ice-cold physiological saline. The brains were then recovered and cut into 4-mm-thick coronal sections using an ice-cold brain mold. Multiple brain tissue punches were taken on both sides of the brain from a single section through the caudate (10-15 punches), putamen (10 punches) and accumbens (5 punches) for dopamine, homovanillic acid (HVA) and 3,4-dihydroxy-phenylacetic acid (DOPAC) measurements by HPLC (Cass, 1996). To assess for regional effects of r-metHuGDNF, the left and right striatum in the coronal brain section used for tissue punches were each divided into three regions of approximately 4 mm each (medial, intermediate, lateral), extending from the lateral ventricle to the lateral border of the putamen (FIG. 2j). All the punches taken in a given region were averaged, providing a single measure per region per animal for dopamine, DOPAC or HVA. For each hemisphere, independent samples t-tests were used to estimate differences in dopamine, DOPAC or HVA levels between animals in the control and r-metHuGDNF treatment groups (assuming unequal variances). Five tissue punches were also taken from a section of the globus pallidus on both sides of the brain for similar analyses.

Intact 4-mm-thick coronal striatal sections, along with the entire midbrain, were immersion-fixed in a 4% paraformaldehyde solution at 4° C. and processed so that 40-μm-thick sections could be cut on a sliding knife microtome through the striatum and the substantia nigra. As described elsewhere (Gash et al., 1995), the sections were further processed for immunohistochemical staining for TH (monoclonal antibody, 1: 1000; Chemicon International Inc., Temecula, Calif.). The number and perikaryal size of TH+ midbrain dopaminergic neurons were estimated using an optical fractionator method for unbiased stereological cell counting (Gash et al., 1996). The ventral tegmental area was not included in the analysis. For each hemisphere, independent samples t-tests were used to analyze the effects of r-metHuGDNF on nigral cell counts or cell size between animals in the control and r-metHuGDNF treatment groups (assuming unequal variances). In addition, a quantitative analysis of TH+ fiber density was conducted on both sides of the brain on a 40-μm-thick section of the striatum. Using the lateral ventricle as the reference point, a 1.2×1.2 mm grid was used to quantify (number of pixels, Bioquant Image Analysis System) the striatal section dorso-ventrally. All the data measured dorso-ventrally at a given distance from the lateral ventricle were averaged, providing a single measure per 1.2-mm-wide dorso-ventral area per animal. The data were analyzed using an analysis of variance (ANOVA) testing for a within-subject factor of distance from the lateral ventricle and a between-subject factor of treatment group (vehicle vs. r-metHuGDNF). The ANOVA was followed by independent samples t-tests (assuming unequal variances). The initial ANOVA testing for within-subject factors of hemisphere (left vs right) and region (medial, intermediate, lateral) and between-subject factors of treatment group (vehicle vs r-metHuGDNF) and route of infusion (intraventricular vs intraputamenal) revealed no main effect for route of infusion for the following measures: TH+ fiber density, nigral cell size and number, dopamine and dopamine metabolite levels in the striatum and globus pallidus. Similarly, non-parametric Mann Whitney-U tests indicated no effect for route of infusion on the weekly behavioral scores of each treatment group. Thus, in accordance with previously published data (Gash et al., 1996), all r-metHuGDNF recipients were treated as one group. P≦0.05 was considered significant in all analyses. In addition to the mixed-group data analysis, data from each animal are presented for behavior, neurochemistry and histology.

r-metHuGDNF Stability and Dose Levels

The nominal concentration of r-metHuGDNF in the pumps was 25 μg/mL. In residual r-metHuGDNF solutions removed at the four, eight and twelve week time points of r-metHuGDNF infusion, r-metHuGDNF levels of 10.0, 18.3 and 14.5 μg/mL, respectively, were measured by ELISA. Separate measurements showed that the sampling technique (adsorption to the syringe used to recover r-metHuGDNF from the pump and vials used for storage) accounted for the loss of 3.2 μg/mL r-metHuGDNF. After adding this back to the ELISA measurements, estimated levels of r-metHuGDNF after four weeks in the pumps at 37° C. ranged from 56-86% of the nominal value. Reverse phase-HPLC showed that 94% of the residual protein after four weeks in a pump eluted in the GDNF native peak, suggesting that the loss was primarily due to adsorption, with little degradation of the remaining protein. Based on average ELISA measurements of 71% of nominal levels and 94% levels of native protein, the nominal daily doses of 7.5 μg/day and 22.5 μg/day were estimated to conservatively represent a minimum of 5 μg/day and 15 μg/day of r-metHuGDNF infused into the brain, respectively.

Parkinsonian Features and the Nigrostriatal System Pre-GDNF Treatment

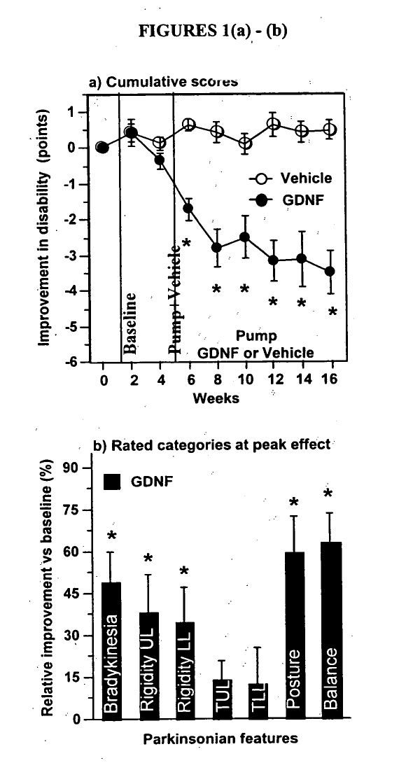

At 45 days post-MPTP administration, the animals were assigned to either r-metHuGDNF (n=8) or vehicle (n=5) test groups so that groups were comparable in their cumulative disability scores prior to catheter implantation (FIG. 1a). To better reflect the improvement in disability, the data were expressed as differences in points between the MPTP/baseline score and post-treatment scores. The parkinsonian features of all thirteen monkeys remained stable (no significant changes) from 45-60 days post-MPTP administration (time 0, FIG. 1a). The catheters were implanted at the 60-day time point. All animals recovered without incident following catheter(s) placement and no fatalities were seen during the four-month study. No significant behavioral changes were seen through the four-week period of vehicle infusion (Table 2). The parkinsonian features then continued to be expressed at the same level in the five vehicle recipients for the remaining three months of the study.

In the vehicle-only recipients at the termination of the study, overall tissue levels of dopamine in the severely lesioned right striatum were 3% of the levels on the left side of the brain (FIG. 2), with the highest residual levels (11%) in the medial striatal region consisting of the periventricular caudate nucleus and the nucleus accumbens (Table 3). The lowest dopamine levels (<1%) were in the intermediate and lateral regions of the striatum containing the putamen.

Dopamine metabolite levels, DOPAC and HVA, were also highest in the medial striatum (Table 3). MPTP also significantly decreased pallidal levels of dopamine and related metabolites in the vehicle-treated animals. When compared to tissue levels in the left pallidum, an average 80% depletion in dopamine (194±16 vs 38±7), HVA (91±11 vs 20±7) and DOPAC (9003±990 vs 1880±250) was seen in the right pallidum of vehicle recipients.

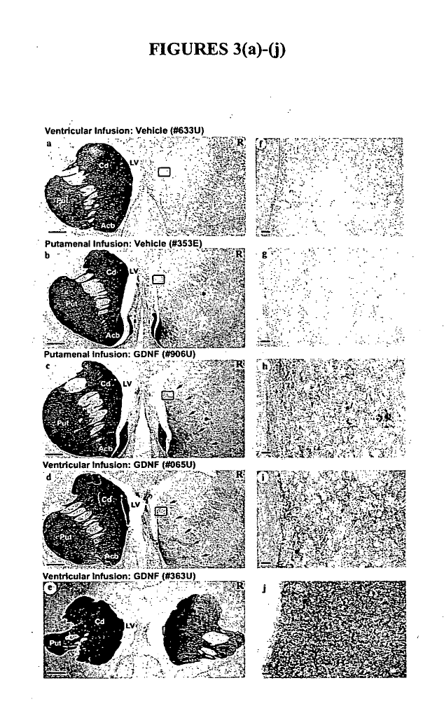

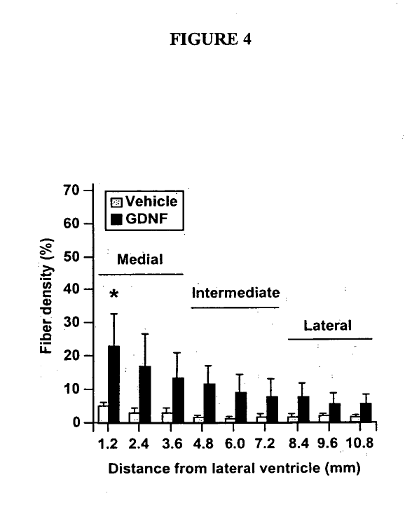

The medial to lateral profile of TH+ fiber density in the right striatum of vehicle recipients mirrored dopamine levels (FIGS. 3a, 3b and 4). Overall right striatal TH+ fiber staining was 3% of levels in the left striatum. The highest fiber density levels (up to 8%) were in the striatal region directly adjacent to the ventricle, with the lowest levels (˜2%) found distal to the ventricle. Consistent with the TH+ fiber reduction in the right striatum, the number of TH+ neurons in the right substantia nigra of vehicle recipients was reduced to 17.5% that found on the contralateral side and the neurons were approximately 100 μm2 smaller.

Anti-Parkinsonian Effects of r-metHuGDNF Infusion

While the chronic infusion of vehicle had no effect on motor functions, a steady improvement was observed in the treated animals during the first month post r-metHuGDNF infusion, reaching an averaged 2.5 points on the rating scale (FIG. 1a). This level of behavioral improvement was thereafter maintained for two additional months, averaging 3.5 points by the end of the study. This represents an overall 36% improvement in disability. In the treated animals, consistent improvements of up to 60% were evident in bradykinesia, rigidity, balance and posture at peak effect (FIG. 1b). Rigidity was defined as decrease in limb extension and/or use (Ovadia et al., 1995). The chronic infusion of r-metHuGDNF was well tolerated by all animals, as it did not induce any observable adverse effects such as dyskinesias, self-mutilation or vomiting. Body weight loss, a side effect observed with acute injections of r-metHuGDNF (Gash et al., 1996), was not significant in the chronic r-metHuGDNF recipients during the 4-month study (data not shown).

Upregulation of the Nigrostriatal Dopaminergic System Post-GDNF Treatment

In comparison to the vehicle recipients, dopamine and its metabolite DOPAC were significantly increased 233% and 180%, respectively, in the periventricular striatum on the lesioned right side of the treated animals (FIGS. 2a and 2d). However, the effects from r-metHuGDNF treatment varied in the more denervated intermediate and lateral portions of the right striatum. On the left side, HVA levels were significantly increased 72%, 70%, and 73% in the periventricular, intermediate and lateral portions of the striatum, respectively, in the r-metHuGDNF recipients (FIGS. 2g-i). When compared to tissue levels in the lesioned right pallidum of vehicle recipients, dopamine, DOPAC and HVA levels were significantly increased 155%, 190% and 47%, respectively, in the right pallidum of the r-metHuGDNF recipients. Only HVA levels were significantly increased 67% in the left globus pallidus of the r-metHuGDNF recipients.

As seen in the vehicle recipients, the direct infusion of MPTP through the right carotid artery almost eliminated TH+ fibers in the right striatum, but largely spared those in the left striatum (FIGS. 3a and b). While a few residual TH+ fibers could be identified at high power magnification in the right striatum of vehicle recipients (FIGS. 3f and g) there was an increase in TH+fibers in the periventricular striatal region of animals receiving infusions of r-metHuGDNF (arrows, FIG. 3h, i and j). A quantitative analysis of TH+ fibers present in the striatum revealed a five-fold increase in TH+ fiber density in the periventricular region of the right striatum compared to vehicle recipients (FIG. 4). TH+ fiber density was most evident along the ventricular border of the striatum and gradually faded in a gradient from the ventricle to deeper into the parenchyma (p<0.025, linear regression analysis). As evident in both the photomicrographs (FIG. 3) and quantitative measurements (FIG. 4) of striatal dopamine fibers, the periventricular GDNF response ranged from moderate increases in TH+ fibers in some animals to a dense fiber network in the periventricular striatum of other monkeys. In contrast to the right striatum, there were no significant differences in fiber density between r-metHuGDNF and vehicle recipients in the left striatum.

However, the effects of r-metHuGDNF on nigral dopamine neurons were bilateral. The number of dopaminergic neurons expressing TH, was significantly increased by more than 20% on both the left side and the lesioned right side.

Similarly, dopaminergic neuron perikaryal size was significantly increased by more than 30% in the left and right substantia nigra.

Conclusions

The chronic infusion of 5 or 15 μg/day r-metHuGDNF into either the lateral ventricle or the striatum of the advanced parkinsonian brain promotes restoration of the nigrostriatal dopaminergic system and significantly improves motor functions in rhesus monkeys. The behavioral improvements were as good or better than previously seen in the same model system in response to levodopa, without inducing side-effects associated with chronic levodopa administration (Myioshi et al., 1997). The functional improvements were associated with a pronounced up-regulation and regeneration of nigral dopamine neurons and their processes innervating the striatum. This was evidenced by 1)>20% bilateral increase in the number of nigral neurons expressing the dopamine marker TH, 2) >30% bilateral increase in nigral dopamine neuronal cell size, 3)>70% and >50% bilateral increase in dopamine metabolite levels in the striatum and the pallidum, 4) 233% and 155% increase in dopamine levels in the periventricular striatal region and in the globus pallidus, respectively, on the lesioned side, and 5) a five-fold increase in periventricular striatal TH+fiber density, on the lesioned right side.

The only other chronic delivery study of GDNF in nonhuman primates has been reported by Kordower et al. (2000) using a lentiviral vector to transfect the GDNF gene into the striatum and substantia nigra of aged and parkinsonian rhesus monkeys. They interpreted their results in parkinsonian animals, which were similar to ours, as demonstrating neuroprotection since GDNF was delivered one week after MPTP toxicity. However, both restorative and protective actions of GDNF may have been involved as the injury sequelae to MPTP are still unfolding in the weeks immediately after MPTP treatment (Herkenham et al., 1991). The GDNF levels produced by transfected cells in their study were not clear, although striatal levels of GDNF were measurable by ELISA eight months after lentiviral GDNF delivery (Kordower et al., 2000). In the present study, the results are primarily attributable to the restorative actions of GDNF, as GDNF was not administered until three months following MPTP-induced nigrostriatal injury, a time at which parkinsonian features are stably expressed (Bankiewicz et al., 1983; Smith et al., 1993).

In the vehicle recipients, the highest levels of dopamine fibers and dopamine were present in the periventricular region of the striatum, the area where significant dopamine fiber regeneration and increases in dopamine levels were found in the r-metHuGDNF recipients. Thus, the effect of r-metHuGDNF on the lesioned right side of the striatum occurred where surviving elements of the nigrostriatal fibers were concentrated. The actions of r-metHuGDNF in restoring striatal dopaminergic innervation may be one of the principle components of the recovery seen in the present study.

The substantia nigra is also an important structure in regulating motor functions. Consistent with previous studies with animal models of PD (Tomac et al., 1995a; Gash et al., 1996), Applicants observed pronounced bilateral changes in nigral dopamine neurons from the chronic infusion of r-metHuGDNF, suggesting an effect on presumably normal nigral neurons on the non-lesioned left side. These effects, along with the bilateral increase in striatal and pallidal dopamine metabolite levels, support the widespread distribution of intracerebrally injected GDNF and are consistent with the diffusion and/or retrograde transport of GDNF from the lateral ventricle or the striatum to the substantia nigra in both rats and monkeys (Tomac et al., 1995b; Lapchak et al., 1998). Evidence suggests that a loss of dopaminergic phenotype precedes the death of nigral neurons in animal models of PD, resulting in quiescent or atrophic dopaminergic neurons (Bowenkamp et al., 1996). Thus, a GDNF-responsive cell population that cannot be visualized using markers such as TH, might have been present and served as the target for GDNF actions within the brain.

### Example 2

### Treatment of Parkinson's Disease in Humans with Direct Infusion of GDNF into the Putamen for 24 Months

Study Design

Five patients with idiopathic, L-dopa responsive PD who were poorly controlled on optimal medical therapy were identified. The first patient (P1) had predominantly unilateral disease affecting the left side and underwent contralateral putamenal implantation of catheter and pump for r-metHuGDNF delivery. The remaining patients (P2-5) had bilateral disease and bilateral putamenal implantations of delivery systems. The precise region of the dorsal putamen to be targeted for infusion was determined by co-localization studies using ¹⁸F-dopa PET and MR images. Under MRI guidance, single-port catheters were placed into the dorsal putamen, the site of maximal loss of ¹⁸F-fluorodopa signal (confirmed by PET in all subjects). Pump placement and stereotactic surgery were tolerated well by all patients. However, there were some complications. P1 required perioperative repositioning of the catheter to center exactly within the dorsal putamen. This was achieved successfully on a second pass during the surgical procedure. In addition, patient 4 developed a wound infection related to the pumps and connection tubing that was successfully treated with explantation of the extracranial devices, antibiotics and re-implantation within 4 weeks.

Patients

Five PD patients were included in this pilot study. Ethical approval was obtained from the local ethics committees both at Frenchay Hospital and the Hammersmith Hospitals Trusts and all participants signed full consent forms. All patients were diagnosed as suffering from idiopathic PD according to standard criteria (brain bank criteria). Patients were selected for surgery when they were suffering significant functional impairment despite optimal medical therapy. Exclusion criteria included women of child-bearing age, age over 65, the presence of clinically significant depression, or systemic disease or inability or unwillingness to comply with long-term follow-up.

Surgery

Sub cortical nuclei were localized and targeted using high-resolution MR images acquired under strict stereotactic conditions. Under general anesthesia, a modified Leksell stereotactic frame was affixed parallel to the orbito-meatal plane. The anterior (AC) and posterior (PC) commisures were identified in a mid-sagittal planning scan. Axial images 2 mm thick were acquired parallel to the AC-PC plane and coronal images orthogonal to these then obtained. Using magnified hard copies of the MRI scans the inversion recovery scans were overlaid with the inverted T2 images to enhance the definition of the putamenal boundaries in both planes. Using the PET images, the area of the postero-dorsal putamen with the lowest ¹⁸F-dopa uptake was targeted for infusion; stereotactic target coordinates were recorded and a trajectory planned. The following day, surgery was performed under general anesthesia. Under stereotactic conditions, 1 mm diameter guide tubes were implanted to a point above the putamen target over a guide rod. A 0.6 mm guide wire was introduced down the guide tube to target, following which the patients underwent repeat MR/CT imaging to verify target localization. The guide wire was then replaced with a 0.6 mm diameter catheter introduced to target. r-metHuGDNF primed SynchroMed pumps (Medtronic Inc, Minneapolis) were implanted in the upper abdominal region, subcutaneously in the first patient, and subfascially (beneath the anterior rectus sheath) in the subsequent cases; subfascial placement reduced the pump profile in the abdomen and improved cosmetic appearance. Catheters were tunneled connecting the pumps to the indwelling 0.6 mm intraparenchymal brain catheters.

The surgical procedure was well tolerated, with only a single-serious treatment-associated adverse event, a pericatheter and peripump infection that required antibiotic treatment and reimplantation.

r-metHuGDNF Production and Infusion

Recombinant-methionyl human glial cell line-derived neurotrophic factor (r-metHuGDNF) was prepared by Amgen Inc. This protein was produced in Escherichia coli cells that contain an expression plasmid with a DNA insert encoding mature human GDNF, with an addition of an amino terminal methionine. r-metHuGDNF is liquid formulated with 10 mM sodium citrate and 150 mM sodium chloride at a pH of 5.0. It was supplied in single-use vials at a concentration of 10 mg/mL. Following implantation, the SynchroMed pumps were programmed to deliver a continuous infusion of 14.4 μg of r-metHuGDNF per putamen per day at rate of 6 μl per hour. The pumps were refilled monthly with fresh solution. The low concentration of r-metHuGDNF was maintained for a period of 8 weeks. At 2 months the pumps were refilled with fresh solution of higher concentration and programmed to deliver 43.2 μg of r-metHuGDNF per putamen per day at a rate of 6 μl per hour. Providing good tolerance and no side effects, this dose was to be maintained for the duration of the trial. However, due to the development of high-signal MRI changes of uncertain significance, the infusion parameters were altered to deliver lower doses (10.8-14.4 μg of r-metHuGDNF) at lower rates (2-6 μl per hour), in attempt to establish safe and clinically effective parameters, with repeat MRI monitoring at regular intervals. Between 12 and 18 months, all patients received a continuous infusion of 14.4 μg of r-metHuGDNF per putamen per day at rate of 6 μl per hour.

At 18 months the dose of GDNF was increased to 28.8 μg per putamen per day at rate of 6 μl per hour, and remained so until 24 months except in P4 who reverted back to 14.4 μg at 20 months.

Clinical Evaluation and Follow-Up

Clinical evaluations were based on the Core Assessment Program for Intracerebral Transplantations (CAPIT)(Langston, J. W. et al., 1992), a validated protocol for evaluating surgical treatments of idiopathic PD. All patients were evaluated on the Unified Parkinson's Disease Rating Scale (UPDRS) and underwent timed motor tests at baseline, 3, 6, 12, 18 and 24 months. Assessments were performed in both off and on medication states. Before they were assessed off medication, patients fasted and medications were withdrawn overnight. The same assessments were then repeated after administration of L-dopa when the patients were “on”.

Health-Related Quality of Life Outcome Measurement and Follow-Up

Patients were also assessed using validated quality of life questionnaires: the 39-item Parkinson's Disease Questionnaire (PDQ 39) and the 36-item Medical Outcomes Study short form health survey (SF-36) were used before surgery and after 3, 6, 12, 18, and 24 months. Descriptive statistics (mean, standard deviation, range, 95% confidence interval) were obtained for each variable. Comparisons over time were made using Student's paired-samples t test.

Neuropsychological Evaluation and Follow-up

Also evaluated were changes in medication (L-dopa equivalents) requirement and neuropsychology, which contained tests of verbal intellect, verbal and visual memory, attention, executive function, anxiety and depression as has been previously described (McCarter, R. J., et al., 2000). The battery of cognitive tests used was designed to minimize the possible confounding effects of both slowness of movement and movement difficulty on cognitive test results. Friedman's Related Samples test was used to evaluate the significance of change over time in the rating scores. All analyses were performed in SPSS. Four of the patients (all of the bilateral cases) underwent pre-operative neuropsychological assessment. All five patients were assessed at 12 and 24 months post implantation. The significance of changes in cognitive test performance was evaluated using confidence intervals derived from the standard error of prediction (Lord and Novack, 1968; Atkinson, L., 1991) around the predicted true score at baseline. A significant change was inferred if a score at either 12 or 24 months fell outside of the confidence interval of the baseline score (for the unilateral case the baseline score was at 12 months and change scores were inferred for performance at 24 months). In addition, a PD control group consisting of 18 patients who had undergone other forms of surgery for PD was used to establish the effect of repeat cognitive assessment over a 12 month period. For each patient in this group two postoperative neuropsychological assessments were available. This control group was comparable with the GDNF patient group in terms of years of education, age at surgery, duration of PD at surgery and NART estimated FSIQ (p>0.05).

Scanning Procedures and Image Analysis

¹⁸F-dopa PET provides a measure of synaptic amino acid decarboxylase (AADC) activity and hence acts as an in vivo marker of dopamine storage and the functional integrity of dopamine terminals. Previous human and animal lesion studies have demonstrated that striatal ¹⁸F-dopa PET correlates with nigral cell numbers, dopamine content in striatal terminals (Garnett et al., 1983; Martin et al., 1989; Brooks et al., 1990(b); Pate et al., 1993) and the UPDRS off medication (Morrish, et al., 1998), in particular with the bradykinesia and rigidity sub scores (Otsuka, et al., 1996). Furthermore, it is possible to demonstrate progressive decline of striatal ¹⁸F-dopa uptake in patients with PD over time (Morrish, et al., 1998; Morrish, et al., 1996). ¹⁸F-dopa PET was used here to assess striatal dopamine terminal function in five PD patients receiving chronic intra-striatal GDNF infusions.

The patients had ¹⁸F-dopa PET pre-operatively, and at 6, 12, 18, and 24 months postoperatively using an ECAT EXACT HR++ camera (CTI/Siemens 966; Knoxville, Tenn.) in 3D acquisition mode following withdrawal from medication for at least 12 hours. Patients received 150 mg of carbidopa and 400 mg of entacapone; 1 hour later 111 MBq of ¹⁸F-dopa in normal saline was administered as an intravenous bolus at the start of scanning. The images were acquired in 3D mode as 26 time frames over 94.5 minutes (1×30 seconds, 4×1 min, 3×2 min, 3×3 min and 15×5 mins). Parametric images of ¹⁸F-dopa influx constants (Ki) were generated from time frames 25.5 to 94.5 minutes post injection using in house software (Brooks, D. J. et al., 1990; Rakshi, J. S. et al., 1999) based on the MTGA approach of Patlak and Blasberg (Patlak, C. S. & Blasberg, R. G., 1985)). Occipital counts from the same time frames were used to generate the tissue reference input function. Integrated images (time frames 25.5-94.5) were used to identify the parameters required to transform the Ki images into standard stereotaxic MNI space. The transformation matrix was then applied to the Ki images. After normalization a gaussian filter (6×6×6 mm) was applied. Mean voxel values of the normalized Ki images were compared throughout the midbrain and basal ganglia at baseline, 6, and 12 months postoperatively using a paired-Student's t-test in SPM99 after application of a mask to eliminate cortical signals and so reduce the number of statistical comparisons. Any regional increases in ¹⁸F-dopa uptake could subsequently be defined as a volume of interest and the mean Ki values for those volumes extracted using the appropriate SPM tool (Brett, M., et al., 2002).

The integrated images were subsequently co-registered to each patient's MRI scan for region of interest (ROI) analysis. All MRIs had been previously reformatted in the AC-PC plane. The subsequent transformation matrix was then applied to individual Ki images in order to transform them into the individual MRI space. Regions of interest (ROIs) were traced on the MRI and included the head of the caudate and the dorsal putamen which was divided into anterior and posterior halves. The position of the catheter tip was also calculated relative to the AC-PC line and an oval region of interest (6 mm×12 mm) centred at the tip location in the axial plane. The ROI was then copied onto 2 planes either side of the slice containing the calculated tip location, creating a 12 mm×6 mm×5 mm (0.36 cc) volume of interest centered on the catheter tip. The regions of interest were then used to sample ¹⁸F activity on the parametric image. In the 4 patients operated on bilaterally, the mean Ki values for the left and right regions of interest were averaged to produce one Ki value for each of the five ROIs per scan. Only the ROIs from the operated right side of the patient who received unilateral surgery were included in the analyses. The patient's Ki values were then subjected to a paired Student's two-tailed t test.

Lack of Adverse Effects of r-metHuGDNF Infusion

Surprisingly, side effects due to r-metHuGDNF infusion itself were very limited. There was no nausea, anorexia, vomiting or weight loss reported as in the previous intraventricular trial (Kordower, J. H. et al., 1999). There were no haematological or blood chemistry abnormalities. At the high dose (43.2 μg/putamen/day), 3 subjects reported abnormalities of taste and smell (soapy or metallic), 2 subjects reported that their dreams had become abnormally vivid, and 4 subjects described a Lhermitte's phenomenon (tingling passing from the neck down through the arms and sometimes onto the trunk and down the legs provoked by neck flexion). None of these effects necessitated immediate dose cessation or reduction, and all remitted (except for occasional, mild Lhermitte's sign) after the dose change discussed below that was instituted after the appearance of a high-density signal on MRI (end of month 3). The Lhermitte's events were mild, intermittent, non-distressing; and most frequently occurred at the higher dose; and in fact it was often described as “pleasurable”.

In all patients, T2 MR images showed a region of high-signal intensity around the tips of the catheters. This response varied between patients, and even between the two hemispheres in bilaterally implanted cases. The signal change was most evident following the dose escalation of r-metHuGDNF. Uncertainty as to the relevance of these changes, led to a reduction of r-metHuGDNF delivery back to 14.4 μg/putamen/day for all patients between 3 and 6 months that resulted in a substantial reduction of the high signal.

Efficacy of r-metHuGDNF Infusion

Improvement in patients' parkinsonian symptoms and signs were evident within 3 months of commencing the infusion and continued to improve throughout the study. Patient diaries revealed that periods of severe immobility, one of the cardinal features of PD that occupied approximately 20% of the waking day prior to surgery, were eliminated completely by 6 months of r-metHuGDNF infusion. At 24-months dyskinesias were reduced significantly by 73% in duration (p<0.05) and were all reported as mild in nature (Table 3). We observed mild dyskinesias in the practically defined off state in P4, who additionally reported short-lived infrequent early morning occurrences. These changes were not due to increases in medication. The study protocol aimed to maintain medication unchanged throughout the first year of r-metHuGDNF treatment. However, P3 had been taking medication on demand due to frequent periods of akinesia at the onset of the study, and needed to reduce his medication as his symptoms improved (Table 7). Patient P5 had increased sensitivity to L-dopa after GDNF infusion and also needed to reduce his dosage. The other 3 patients needed slight increases in their L-dopa equivalents intake. At 24 months, the mean daily dose of L-dopa equivalents was reduced by 26% (based on a formula as designated by Pahwa, et al., (1997)).

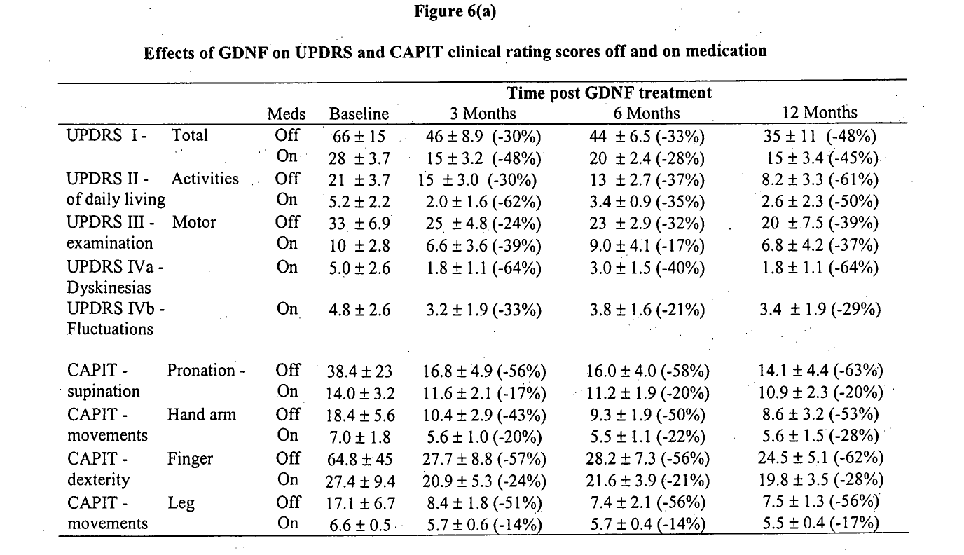

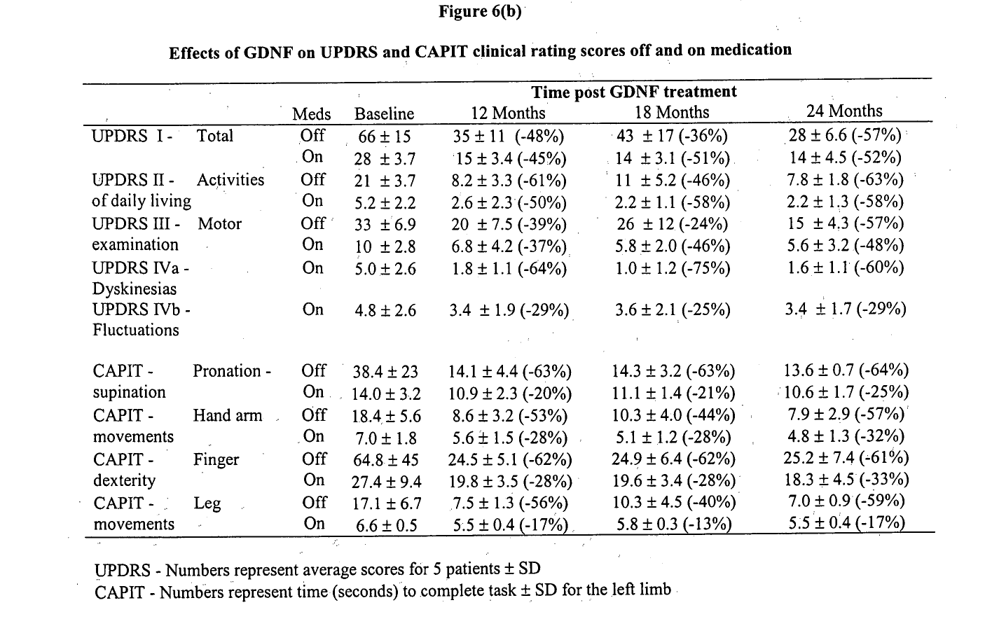

The most widely used and validated scale for assessing functional changes in PD is the Unified Parkinson's Disease Rating Scale (UPDRS). In all patients, the rate of symptomatic improvement was maximal in the first 3 months of GDNF infusion and, thereafter, there was slower but sustained improvement throughout the entire 24 months of follow-up. The total UPDRS scores in the clinically defined “off” phase when assessed 12 months following commencement of r-metHuGDNF infusion showed a reduction from baseline of 48% and (FIG. 6a). Although this was a small group of patients, we performed a non-parametric significance test, which showed that this reduction was highly significant across the three time points (P<0.005; Friedman.test). The largest effects were seen over the first three months, but the effect persisted throughout the trial. There was also a 45% reduction in total UPDRS scores in the clinically defined “on” phase by 12 months, which followed a similar pattern over time (P<0.002; Friedman test; FIG. 6a). Although P4's final score was still below baseline (FIG. 6a), he did show a worsening of symptoms at the twelve-month assessment; this may have been due to an unrelated inter-current infection.

The patients experienced a mean 41% (p<0.001) improvement in ‘off-period’ total UPDRS over the first 12 months of follow-up. When the results were broken down, it was clear that these effects in total UPDRS during the “off” phase were reflected by improvements in both activities of daily living (ADL) UPDRS subscale II (P<0.002; Friedman test; FIG. 6a) and motor UPDRS subscale III scores (P<0.002; Friedman test; FIG. 6a). The patients experienced a mean 44% (p<0.0001) improvement in ‘off-period’ part III motor UPDRS score.

The symptomatic effect at 18-months showed some deterioration from 12 months so the GDNF infusion dose was increased. At 24 months, there was progressive benefit in comparison to 12 months, with the scores in the off-medication state tending towards the baseline best on-medication state. The overall UPDRS scores progressively improved by 57% (p<0.005) and 52% (p<0.005) in both the off and on medication states respectively (FIG. 6a,b). The effect of 24 month GDNF infusion on the patients' motor performance (UPDRS part III) was significant in both the off and on medication states, resulting in a 57% (p<0.001) and 48% (p<0.01) score reduction, respectively (FIG. 6b). There was significant improvement in the patients' functional performance with GDNF infusion, as demonstrated by activities of daily living scores (UPDRS part II), which at 24 months were reduced by 63% (p<0.005) and 58% (P<0.05) in the off and on medication states respectively (FIG. 6b).

Involuntary movements (i.e. dyskinesias) are a common problem in PD and were suffered by all but one of the patients at the start of the trial. The overall dyskinesia scores (UPDRS subscale IVa) were significantly reduced on medication following r-metHuGDNF infusion for 12 months (P<0.01; Friedman test). No dyskinesias were seen in these patients when off medication. Timed motor tests were assessed and followed the protocol outlined by the Core Assessment Program for Intracerebral Transplantation (CAPIT) (Langston, J. W. et al., 1992). These were also improved in both the “off” and “on” medication states. All the timed motor tests showed significant improvements with GDNF infusion in the off medication state (FIG. 6a, b). One patient was unable to complete the stand-walk test without multiple freezing episodes and support whilst in the off medication state preoperatively, but was able to accomplish the task without freezing and support after 12 months of GDNF infusion whilst in the off medication state. Long-term infusion resulted in progressively improved scores for akinesia, rigidity and tremor, impairment of arising from chair, gait and postural instability, when patients were evaluated off medication. GDNF infusion reduced levodopa-induced dyskinesias and motor fluctuations based on complications of therapy scores (UPDRS part IV), which at 24 months were improved by 60% and 29% respectively (FIG. 6b).

Effect of GDNF Infusion on Quality of Life Measures

The functional status of the patients was assessed using the Parkinson's Disease Questionnaire (PDQ-39) (Peto, V., 1995) and the 36-item Medical Outcomes Study short form health survey (SF-36) before surgery and after 3, 6, 12, 18, and 24 months. All PDQ-39 domains showed improvement over time.

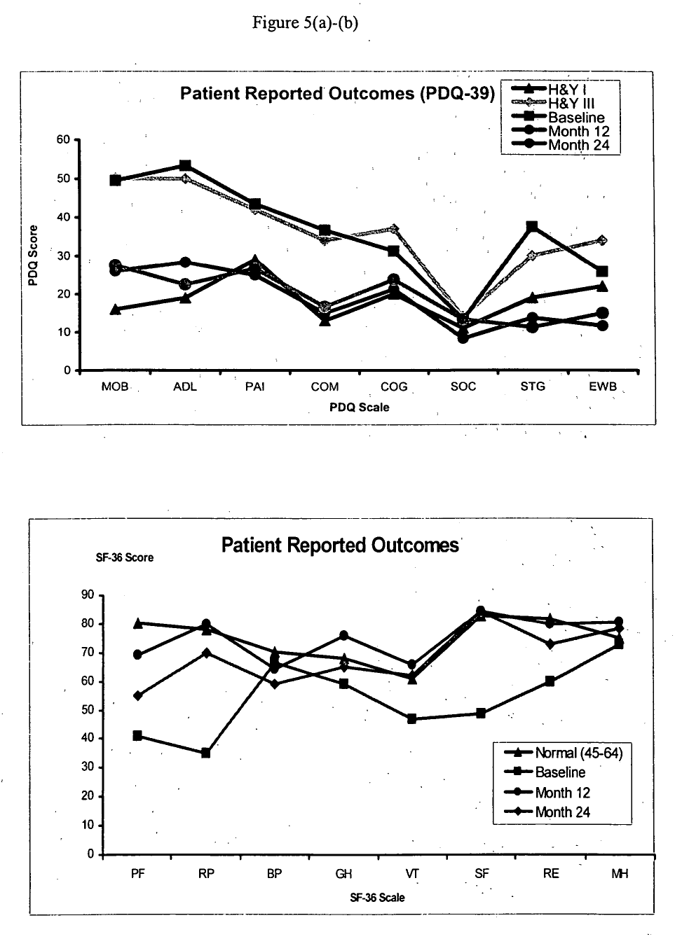

At baseline, the scores were similar to a control PD population with moderate disease (Hoehn and Yahr III) and at 12 and 24 months these scores tended towards a control population with mild disease (Hoehn and Yahr I) (FIG. 5a). At 12 months all dimensions were improved, with bodily discomfort significantly improved (p<0.05). At 24 months all dimensions except social support were improved, with significant improvements for activities of daily living, stigma and cognition (p<0.05) (FIG. 5a). All SF-36 domains were improved at both 12 and 24 months.

With time the scores improved towards those for an age-matched healthy population (FIG. 5b). At 12 months scores for physical functioning and vitality were significantly improved (p<0.05) (FIG. 5b).

Effect of GDNF Infusion on Neuropsychological Measures

The test-retest performance of the PD control group over a 12 month period was assessed using repeated measures ‘t’ tests. No significant improvement in test performance was observed. Significant declines in mean test performance over the 12 month period were observed for the arithmetic subtest of the WAIS-R, immediate and delayed recall of a short story, RAVLT learning and the number of errors obtained on the Tower of London test.

Using the 90% confidence interval of each GDNF patient's baseline score it was found that the majority of patient test scores remain unchanged both at 12 and 24 months. At 12 months there were, however, two significantly improved test scores at the 90% confidence level, one on VIQ and the other on RAVLT learning. These declines occurred in different patients. In addition at 12 months there were two significantly improved test scores, one on VIQ and the other on the delayed recall of a story. Again these significant changes occurred in two different patients. At 24 months there were more significant positive changes in test performance than at 12 months. As with 12 months there were also two test scores that declined, one on VIQ and the other on RAVLT learning. Again these declines occurred in two different patients. The declined VIQ at 24 months was in the same patient who demonstrated a significant decline at 12 months.

The significant improvements in test performance at 24 months occurred on VIQ, immediate and delayed short story recall, RAVLT learning and short delayed recall of the RAVLT. Many of these significant improvements were also evident at the 95% confidence interval. Of these changes improvement in the immediate and delayed recall of a story occurred in one patient and improvement in delayed recall of a story in another patient. One patient demonstrated an improvement in VIQ. Two patients demonstrated an improvement in RAVLT learning with one demonstrating an improvement additionally at the short delayed recall of the RAVLT.

¹⁸F-Dopa PET Scan Changes

Positron emission tomography (PET) scans of ¹⁸F-dopa uptake gives a direct indication of dopamine storage within the brain, and has been used extensively to assess dopamine changes in PD (Morrish, P. K., et al., 1996). Baseline scans revealed that the posterior segment of the putamen in all patients had low ¹⁸F-dopa uptake. These regions of reduced dopamine storage were used to establish the optimal site for placing the catheters for r-metHuGDNF delivery. At 6 months r-metHuGDNF was shown to increase ¹⁸F-dopa uptake by 24.5% (0-49%) within a 0.36 cc ovoid volume around the tip of each catheter. At 12 and 18 months post r-metHuGDNF infusion the same analysis also revealed increases in ¹⁸F-dopa uptake. However, this was complicated by the fact that patient 2 moved considerably during the third scan at 12 months which may have resulted in an under estimate of his true ¹⁸F-dopa uptake. The ¹⁸F-dopa increases at 18 months were significant using a Student's two-tailed t-test in regions where such increases were hypothesized (P=0.021).

Although interrogating a single volume around the tip of each catheter may reveal local changes in ¹⁸F-dopa uptake, changes elsewhere in the putamen or in the midbrain would be missed using this technique. Statistical parametric mapping (SPM) localizes significant changes in ¹⁸F-dopa storage between scans throughout the brain and has recently been shown to be a useful method for detecting changes in dopaminergic function (Brooks, D. J. et al., 1990; Rakshi, J.

S. et al., 1999) and for following the progression of PD (Whone, A. L. et al., 2002). When the preoperative and 6 month Ki images were interrogated with SPM three regions demonstrated focal increases in ¹⁸F-dopa uptake—(i) right posterior dorsal putamen (+17.9%), (ii) left medial dorsal putamen (+25.3%) and (iii) right substantia nigra (+16%). The exact locations of the regions with increased ¹⁸F-dopa uptake were identified with SPM superimposed on a mean MRI template constructed from the individual T1 weighted MRIs of the 5 patients. The movement of patient 2 during the 12 month scan again made interpretation of this small sample very difficult. Despite this, the patients as a group continued to demonstrate a significant increase in ¹⁸F-dopa within the right substantia nigra region (+26%; paired t test; P<0.05 uncorrected at cluster level). ¹⁸F-dopa PET: Region of Interest Analysis Repeated measures analysis of variance (MANOVA) identified a significant difference in the ¹⁸F-dopa uptake constant (Ki) between the baseline scan and all post-operative scans in the posterior putamen (p<0.001). The increase was most marked at 24 months (60%, p<0.001). Apost-hoc comparison of the post-operative Ki values using the Tukey-Kramer multiple comparisons test demonstrated significantly higher ¹⁸F-dopa uptake at 24 months than at 6, 12 and 18 months in the posterior putamen (p<0.001 for 6 months vs. 24 months, p<0.05 for 12 months vs. 24 months, p<0.01 for 18 months vs. 24 months). Limiting the region of interest to the volume of putamen surrounding the catheter tip produced similar results but with a greater percentage increase in ¹⁸F-dopa uptake (83% at 24 months). Repeated measures analysis of variance also identified a significant difference in ¹⁸F-dopa uptake in the whole putamen (p=0.0237), the post-hoc analysis however was only able to identify a significant increase between baseline and 24 months. We were unable to identify any significant change in ¹⁸F-dopa uptake in either the head of caudate or the anterior half of dorsal putamen over the course of this study.

F-dopa PET: SPM

Pre-op and 24 month post surgery images of ¹⁸F-dopa uptake constants (Ki maps) were interrogated with SPM99. Two regions showed significant increases in ¹⁸F-dopa uptake: the left posterior dorsal putamen (p<0.0001 cluster corrected, Z score 3.01) and right posterior dorsal putamen (p<0.0001 cluster corrected, Z score 3.74). The right posterior dorsal putamen was also identified as a region of increased ¹⁸F-dopa uptake, albeit at a lower level of significance, when the baseline images were compared with the 6, 12, and 18 month Ki maps. The left posterior dorsal putamen was identified by SPM99 as a region of significantly increased ¹⁸F-dopa uptake when the baseline scans were compared with the 6 and 18 month Ki maps. The early SPM comparisons, i.e. baseline vs. 6 months and baseline vs. 12 months, localised a third region of increased ¹⁸F-dopa uptake involving the right substantia nigra. SPM99 was unable to identify increased ¹⁸F-dopa uptake in the right substantia nigra at 18 or 24 months.

Conclusions

Applicants show for the first time that direct intraputaminal GDNF infusion in patients with PD is safe, can be tolerated for at least two years and appears to effectively treat PD in humans. Furthermore, Applicants show for the first time that direct intraputaminal GDNF infusion in patients leads to sustained increases in dopamine uptake in the putamen. Although L-dopa equivalents were maintained in 3 of the 5 patients throughout this study, there was a significant reduction in dyskinetic movements by over 60% while on medication, and no dyskinetic movements off medication which has also been reported following intracerebral infusion of GDNF in monkeys (Miyoshi, Y. et al. (1997)). This result is in contrast to recent fetal transplant trials where some patients experience dyskinesias of unknown origin when off medication (Freed, C. et al, 2001; Hagell, P. et al., 2002). This finding is of major significance and a surprising benefit for PD patients and suggests that GDNF may act to regulate dopamine production, release and metabolism within the striatum thus allowing for a more physiological processing of motor output. Thus, PD patients administered GDNF directly to the putamen experience a far better quality “on” time than ever reported previously.

The overall 57% reductions in “off” UPDRS scores after 24 months of treatment are surprisingly dramatic. The improvements were progressive, with the “off” period scores at 12 months tending closely towards the baseline best “on” period scores. A decline in most CAPIT timed tests adds further evidence substantiating an overall subjective improvement. The finding of significant reductions in the UPDRS scores in the “on” clinical phase to 52% of baseline is unprecedented. No such improvement in the “on” state following surgical treatment for PD (The Deep-Brain Stimulation for Parkinson's Disease Study Group., New England Journal Medicine 345, 956-963, 2001) or following transplantation of fetal dopamine neurons (Lindvall, 0. & Hagell, P., 2000) has previously been reported.

PD is also often associated with impaired olfaction (Quinn, N. P. et al., 1987) assumed to be the result of Lewy bodies in the olfactory bulb and cortex (Daniel, S. E. et al., 1992). In fact, three patients had long-standing loss of sensation of smell and taste, as is often the case in PD. Surprisingly, three patients reported a return of sense of smell following r-metHuGDNF infusion. These symptoms greatly improved or resolved completely between 3 and 6 weeks of r-metHuGDNF infusion (Table 7). However, with this recovery, abnormal sensations of taste were intermittently experienced, with “metallic” or “soapy” tastes being reported.

At the highest dose of r-metHuGDNF, three patients also reported recovery of normal sexual function, both in terms of interest and potency. This recovery subsided as the dose was reduced.

The patients' functional performance was improved with infusion, as demonstrated by significant improvements in the activities of daily living scores (UPDRS part I and PDQ-39 ADL dimension).

It was apparent from the neuropsychological data that there was no detrimental effect of GDNF infusion on cognition. The cognitive test results in fact provide evidence of an improvement in verbal anterograde memory function in some patients after 24 months of GDNF infusion. The finding of improved memory function at 24 months was not attributable to practice. The comparable control group of 18 PD patients did not show a significant improvement in memory function after an interval of 12 months and in fact demonstrated significant declines on a number of measures of verbal anterograde memory function. It is also unlikely that the observed improvement in memory function is attributable to statistical false positive error. Out of the 54 statistical tests conducted one would expect 5 significant results by chance alone (with p=0.1). At 24 months there were, however, nine significant changes observed, two in the negative direction and seven in the positive direction. Of the positive changes observed six were on measures of anterograde verbal memory function.

In addition to clinical improvements, the continuous infusion of r-methHuGDNF was associated with surprisingly dramatic increases in total putamen ¹⁸F-dopa uptake capacity. Previous studies have estimated the annual decline in putamen ¹⁸F-dopa uptake to be around 10% of the baseline value in PD patients (Morrish, P. K., et al., 1996; Wenning, G. K. et al., 1997). In contrast, we report a 23.5% increase in total putamen ¹⁸F-dopa uptake following a two year infusion of GDNF into the posterior putamen. The increase in total putaminal ¹⁸F-dopa uptake and storage was entirely due to increases in the posterior half of the putamen or, more specifically, the putamen adjacent to the catheter tip (60% and 83%, respectively (p<0.01)). In contrast, there was only a 6.8% increase in ¹⁸F-dopa uptake in the anterior half of the putamen (p=0.223). In addition, in the patient who received GDNF unilaterally, a decline in total putamen ¹⁸F-dopa uptake of 7% over the course of the 2 years of follow up was detected. Thus, it is quite clear from these studies that the diffusion of GDNF in concentrations sufficient to induce significant changes in dopamine terminal function is limited. These results conflict with several previous animal studies in which the unilateral administration of GDNF to a unilaterally MPTP lesioned rhesus monkey resulted in significant increases in dopamine metabolites and nigral cell numbers on the contralateral side (Grondin, et al., 2002; Gash, et al., 1996). The difference between the relatively limited spread of GDNF activity in this study and the more widespread effects of GDNF in previous studies is probably due to brain size. The rhesus monkey brain is 12-15 times smaller than the human brain. As a consequence, diffusion from one hemisphere to another involves a relatively modest distance particularly following intra-nigral administration. The limited diffusion observed in this study is consistent with one recent primate study in which aged rhesus monkeys received unilateral intra-striatal infusions of 22.5 μg of GDNF per 24 hours. This study reported a maximal diffusion distance of 11 mm from the catheter tip. The present study clearly indicates that chronic administration of GDNF to the brain must be localized precisely in order to achieve therapeutic effectiveness in humans.

Increased dopamine storage at the level of the substantia nigra suggest that either local nigral dopamine terminals or neuron cell bodies were also responding to the r-metHuGDNF delivered to putamen nerve terminals possibly via its retrograde transport while the early changes in sense of smell, and the overall reductions in UPDRS at 3 months suggests at least an initial pharmacological action of r-metHuGDNF within the putamen. This is likely, in part, to involve a direct stimulatory effect on dopamine release as shown in rodent models (Hoffman, A. F. et al., 1997).

### REFERENCES

Ai Y, Markesbery W, Zhang Z, Grondin R, Elseberry D, Gerhardt, G A, Gash, D. Intraputamenal infusion of GDNF in aged rhesus monkeys: distribution and dopaminergic effects. Journal of Comparative Neurology. 461:250-261 (2003).

Arenas, E., Åkerud, P., Wong, V., Boylan, C., Persson, H., Lindsay, R. M., Altar, C. A. Effects of BDNF and NT-4/5 on striatonigral neuropeptides or nigral GABA neurons in vivo. Eur. J. Neurosci., 8: 1707-1717 (1996).

Atkinson L. Three standard errors of measurement and the Wechsler Memory Scale—Revised. Psychological Assessment: A journal of consulting and clinical psychology, 3:136-138 (1991).

Bhatia, K. P. & Marsden, C. D. The behavioural and motor consequences of focal lesions of the basal ganglia in man. Brain, 117 (Pt 4), 859-876 (1994).

Bankiewicz K S, Oldfield E H, Chiueh C C, Doppman J L, Jacobowitz D M, Kopin I J. Hemiparkinsonism in monkeys after unilateral internal carotid artery infusion of 1-methyl-4-phenyl-1,2,3,6-tetrahydropyridine. Life Sci., 39: 7-16 (1986).

Bensadoun J-C, Deglon N, Tseng J L, Ridet J L, Zurn A D, Aebischer P. Lentiviral vectors as a gene delivery system in the mouse midbrain: Cellular and behavioral improvements in 6-OHDA model of Parkinson's disease using GDNF. Exp. Neurol., 164: 15-24 (2000).

Bjorklund A, Kirik D, Rosenblad C, Georgievska B, Lundberg C, Mandel R J. Towards a neuroprotective gene therapy for Parkinson's disease: use of adenovirus, AAV and lentivirus vectors for gene transfer of GDNF to the nigrostriatal system in the rat Parkinson model. Brain Research, 886: 82-98 (2000a).

Bjorklund A, Lindvall A. Parkinson's disease gene therapy moves towards the clinic. [Letter]. Nature Med., 6: 1207-1208 (2000b).

Bowenkamp K E, David D, Lapchak P L, Henry M A, Grandholm A C, Hoffer B. J. 6-Hydroxydopamine induced the loss of the dopaminergic phenotype in substantia nigra neurons in the rat: a possible mechanism for restoration of the nigrostriatal circuit mediated by glial cell line-derived neurotrophic factor. Exp. Brain Res., 111: 1-7 (1996).

Brett, M., Anton, J., Valabregue, R., & Poline, J. Region of interest analysis using an SPM toolbox. NeuroImage, 16 (2002).

Brooks, D. J. The relationship between locomotor disability, autonomic dysfunction, and the integrity of the striatal dopaminergic system in patients with multiple system atrophy, pure autonomic failure, and Parkinson's disease, studied with PET. Brain, 113 (Pt 5) 1539-1552 (1990a).

Brooks D J, Ibanez V, Sawle G V, et al. Differing patterns of striatal ¹⁸F-dopa uptake in Parkinson's disease, multiple system atrophy, and progressive supranuclear palsy. [comment]. Annals of Neurology, 28:547-555 (1990b).

Cass W. A. GDNF selectively protects dopamine neurons over serotonin neurons against the neurotoxic effects of methamphetamine. J. Neurosci., 16: 8132-8139 (1996).

Choi-Lundberg D L, Lin Q, Chang Y-N, Chiang Y L, Hay C M, Mohajeri H. Dopaminergic neurons protected from degeneration by GDNF gene therapy. Science, 275: 838-841 (1997).

Connor B, Kozlowski D A, Schaller T, Tillerson J L, Davidson B L, Bohn M C. Differential effects of glial cell line-derived neurotrophic factor (GDNF) in the striatum and substantia nigra of the aged parkinsonian rat. Gene Therapy, 6: 1936-1951 (1999).

Costa S, Mahmoud M, Pearce R K B, Jenner P. Glial cell line-derived neurotrophic factor concentration dependently improves disability and motor activity in MPTP-treated common marmosets. European Journal Pharmacol., 412: 45-50 (2001).

Daniel, S. E. & Hawkes, C. H. Preliminary diagnosis of Parkinson's disease by olfactory bulb pathology. Lancet, 340, 186 (1992).

Emborg-Knott, ME, Domino E F. MPTP-induced hemiparkinsonism in nonhuman primates 6-8 years after a single unilateral intracarotid dose. Exp Neurol., 152: 214-220 (1998).

Freed C. R., Greene P. E., Breeze R. E. Transplantation of embryonic dopamine neurons for severe Parkinson's disease. N. Engl. J. Med., 344:710-719 (2001).

Förander, P., Söderström, S., Humpel, C., Strömberg, I. Chronic infusion of nerve growth factor into rat striatum increases cholinergic markers and inhibits striatal neuronal discharge rate. European Journal of Neuroscience, 8:1822-1832 (1996).

Garnett E S, Fimau G, Nahmias C. Dopamine visualized in the basal ganglia of living man. Nature, 305: 137-138 (1983).

Gash D M, Zhang Z, Cass W A, Ovadia L, Simmerman L, Martin D. Morphological and functional effects of intranigrally administered GDNF in normal rhesus monkeys. J Comp. Neurol., 363: 345-358 (1995).

Gash D M, Zhang Z, Ovadia A, Cass W A, Yi A, Simmerman L. Functional recovery in parkinsonian monkeys treated with GDNF. Nature, 380: 252-255 (1996).

Gash D M, Zhang Z, Gerhardt Ga. Neuroprotective and neurorestorative properties of GDNF. [Review]. Ann Neurol., 44: S121-S125 (1998).

Grondin R, Zhang Z, Elsberry D D, Gerhardt G A, Gash D M. Chronic intracerebral delivery of trophic factors via a programmable pump as a treatment for parkinsonism. In: Mouradian M M, editor. Parkinson's disease: Methods and Protocols. Totowa, N.J.: Humana Press (2001) p. 257-267.

Grondin, R., Zhang; Z., Yi, A., et al. Chronic, controlled GDNF infusion promotes structural and functional recovery in advanced parkinsonian monkeys.[comment]. Brain, 125: 2191-2201 (2002).

Hagell, P. Dyskinesias following neural transplantation in Parkinson's disease. Nat. Neurosci., 5: 627-628 (2002).

Herkenham, M, Little M D, Bankiewicz K, Tang S C, Markey S P, Johannessen J N. Selective retention of MPP+ within the monoaminergic systems of the primate brain following MPTP administration: An in vivo autoradiographic study. Neuroscience, 40: 133-158 (1991).

Hoffman, A. F., van Home, C. G., Eken, S., Hoffer, B. J., & Gerhardt, G. A. In vivo microdialysis studies on somatodendritic dopamine release in the rat substantia nigra: effects of unilateral 6-OHDA lesions and GDNF. Exp. Neurol., 147-130-141 (1997).

Hou, J-G. G., Lin L-F. H., Mytilinenou, C., Glial cell line-derived neurotrophic-factor exerts neurotrophic effects on dopaminergic neurons in vitro and promotes their survival and regrowht after damage by 1-methyl-4-phenylpyridinium. J. Neurochem., 66: 74-82 (1996).

Imbert C, Bezard E, Guitraud S, Boraud T, Gross C E. Comparison of eight clinical rating scales used for the assessment of MPTP-induced parkinsonism in the Macaque monkey. J. Neurosci. Meth, 96: 71-76 (2000).

Jellinger K. Overview of morphological changes in Parkinson's disease. Adv. Neurol. 45 (1986).

Kearns C, Gash, D M. GDNF protects nigral dopamine neurons against 6-hydroxydopamine in vivo. Brain Research, 672:104-111 (1995).

Kirik D, Rosenblad C, Bjorklund A, Mandel R J. Long-term rAAV-mediated gene transfer of GDNF in the rat Parkinson's model: Intrastriatal but not intranigral transduction promotes functional regeneration in the lesioned nigrostriatal system. J. Neurosci., 20: 4686-4700 (2000).

Kirik D, Georgievska B, Rosenblad C, Bjorklund A. Delayed infusion of GDNF promotes recovery of motor function in the partial lesion model of Parkinson's disease. Eur. J. Neurosci., 13: 1589-1599 (2001).

Kish S J, Shannak K, Hornykiewicz O. Uneven pattern of dopamine loss in the striatum of patients with idiopathic Parkinson's disease. Pathophysiology and clinical implications. N. Eng. J. Med., 318: 876-880 (1988).

Kordower J H, Palfi S, Chen E-Y, Shuang Y, Ma Y, Sendera T. Clinicopathological findings following intraventricular glial-derived neurotrophic factor treatment in a patient with Parkinson's disease. Ann. Neurol., 46: 419-424 (1999).

Kordower J H, Emborg M E, Bloch J, Ma S Y, Chu Y, Leventhal L. Neurodegeneration prevented by lentiviral vector delivery of GDNF in primate models of Parkinson's disease. Science, 290: 767-773 (2000).

Lang A E, Lozano A M. Parkinson's disease. First of two parts. [Review]. N. Engl. J. Med., 339:1044-1053 (1998a).

Lang A E, Lozano A M. Parkinson's disease. Second of two parts. [Review]. N. Engl. J. Med., 339: 1130-1143 (1998b).

Langston, J. W. et al. Core assessment program for intracerebral transplantations (CAPIT). Mov Disord., 7, 2-13 (1992).

Lapchak P A, Araujo D M, Hilt D C, Jiao S, Collins F, Miyoshi Y, et al. Topographical distribution of [¹²⁵I]-glial cell line-derived neurotrophic factor in unlesioned and MPTP-lesioned rhesus monkey brain following a bolus intraventricular injection. Brain Res., 789: 9-22 (1998).

Lindner M D, Winn S R, Baetge E E, Hammang J P, Gentile F T, Doherty E, et al. Implantation of encapsulated catecholamine and GDNF-producing cells in rats with unilateral dopamine depletions and parkinsonian symptoms. Exp. Neurol., 132: 62-76 (1995).

Lindvall, O. & Hagell, P. Clinical observations after neural transplantation in Parkinson's disease. Prog. Brain Res., 127: 299-320 (2000).

Lin L-F. H., Doherty D. H., Lile J. D., Bektesh S., Collins F., GDNF: A glial cell line-derived neurotrophic factor for midbrain dopaminergic neurons. Science, 260: 1130-1132 (1993).

Lin L-F. H., Zhang T. J., Collins F., Armes L. G., Purification and initial characterization of rat B49 glial cell line-derived neurotrophic factor. J. Neurochem., 63: 758-768 (1994).

Lord F M, Novick, M. R. Statistical theories of mental test scores. Reading, MA: Addison-Wesley, 1968.

Ma, Y., Feigin, A., Dhawan, V., Fukuda, M., Shi, Q., Greene, P., Breeze, R., Fahn, S., Freed, C., Eidelberg, D. Dyskinesia after fetal cell transplantation for parkinsonism: a PET study (2002).

Martin W R, Palmer M R, Patlak C S, Calne D B. Nigrostriatal function in humans studied with positron emission tomography. Annals of Neurology, 26:535-542 (1989).

McCarter, R. J., Walton, N. H., Rowan, A. F., Gill, S. S., & Palomo, M. Cognitive functioning after subthalamic nucleotomy for refractory Parkinson's disease. J Neurol. Neurosurg. Psychiatry, 69: 60-66 (2000).

Miyoshi Y, Zhang Z, Ovadia A, Lapchak P A, Collins F, Hilt D, et al. Glial cell line-derived neurotrophic factor-levodopa interactions and reduction of side effects in parkinsonian monkeys. Ann. Neurol., 42: 208-214 (1997).

Morrish, P. K., Sawle, G. V., Brooks, D. J. An [¹⁸F]dopa-PET and clinical study of the rate of progression in Parkinson's disease. Brain, 119 (Pt 2), 585-591 (1996).

Morrish, P. K., Rakshi, J. S., Bailey, D. L. Measuring the rate of progression and estimating the preclinical period of Parkinson's disease with [¹⁸F]dopa PET. [comment]. Journal of Neurology, Neurosurgery & Psychiatry, 64: 314-319 (1998).

Olson, L., Nordberg, A., von Holst, H., Backman, T., Ebendal, T., Alafuzoff, I., Amberla, K., Hartvig, P., Herlitz, A., Lilja, A., Lundqvist, H., Langstrom, B., Meyerson, B., Persson, A., Viitanen, M., Winblad, B., and Seiger, A. Nerve growth factor affects ¹¹C-nicotine binding, blood flow, EEG, and verbal episodic memory in an Alzheimer patient (Case Report). Journal of Neural Transmission [P-D Sect], 4: 79-95 (1992).

Olson L. Combating Parkinson's disease-step three. [Review]. Science, 290: 721-724 (2000).

Otsuka, M., Ichiya, Y., Kuwabara, Y., et al. Differences in the reduced ¹⁸F-Dopa uptakes of the caudate and the putamen in Parkinson's disease: correlations with the three main symptoms. Journal of the Neurological Sciences, 136: 169-173 (1996).

Ovadia A, Zhang Z, Gash D M. Increased susceptibility to MPTP toxicity in middle-aged rhesus monkeys. Neurobiol. Aging, 16: 931-937 (1995).

Pate B D, Kawamata T, Yamada T et al. Correlation of striatal fluorodopa uptake in the MPTP monkey with dopaminergic indices. Annals of Neurology, 34: 331-338 (1993).

Patlak, C. S. & Blasberg, R. G. Graphical evaluation of blood-to-brain transfer constants from multiple-time uptake data. Generalizations. J. Cereb. Blood Flow Metab. 5: 584-590 (1985).

Pahwa R, Wilkinson S, Smith D et al. High-frequency stimulation of the globus pallidus for the treatment of Parkinson's disease. Neurology, 49: 249-253 (1997).

Peto, V., Jenkinson, C., Fitzpatrick, R., & Greenhall, R. The development and validation of a short measure of functioning and well being for individuals with Parkinson's disease. Qual. Life Res., 4: 241-248 (1995).

Quinn, N. P., Rossor, M. N., & Marsden, C. D. Olfactory threshold in Parkinson's disease. J. Neurol. Neurosurg. Psychiatry, 50: 88-89 (1987).

Rakshi, J. S. et al. Frontal, midbrain and striatal dopaminergic function in early and advanced Parkinson's disease A 3D [(18)F]dopa-PET study. Brain, 122 (Pt 9): 1637-1650 (1999).

Rosenblad C, Martinez-Serrano A, Bjorklund A. Intrastriatal glial cell line-derived neurotrophic factor promotes sprouting of spared nigrostriatal dopaminergic afferents and induces recovery of function in a rat model of Parkinson's disease. Neuroscience, 82: 129-137 (1998).

Smith R D, Zhang Z, Kurlan R, McDermott M, Gash D M. Developing a stable bilateral model of parkinsonism in rhesus monkeys. Neuroscience, 52: 7-16 (1993).

Smith Y, Kieval J Z. Anatomy of the dopamine system in the basal ganglia. [Review]. Trends Neurosci., 23(Suppl): S28-S33 (2000).

The Deep-Brain Stimulation for Parkinson's Disease Study Group. Deep brain stimulation of the subthalamic nucleus or the pars interna of the globus palidus in Parkinson's Disease. New England Journal Medicine, 345: 956-963 (2001).

Tomac A, Lindqvist E, Lin L-F H, Ogren S O, Young D, Hoffer B J, et al. Protection and repair of the nigrostriatal dopaminergic system by GDNF in vivo. Nature, 373: 335-339 (1995a).

Tomac A, Widenfalk, Lin L-FH, Kohno T, Ebendal T, Hoffer B J, et al. Retrograde axonal transport of glial cell line-derived neurotrophic factor in the adult nigrostriatal system suggests a trophic role in the adult. Proc. Natl. Acad. Sci. USA, 92: 8274-8278 (1995b).

Wenning, G. K. et al. Short- and long-term survival and function of unilateral intrastriatal dopaminergic grafts in Parkinson's disease. Ann. Neurol., 42, 95-107 (1997).

Whone, A. L. et al. The REAL-PET study: Slower progression in early Parkinson's disease treated with ropinirole compared with L-dopa. Neurology, 58, A82-A83 (2002).

Zhang Z., Miyoshi Y, Lapchak P A, Collins F, Hilt D, Lebel C, et al. Dose response to intraventricular glial cell line-derived neurotrophic factor administration in parkinsonian monkeys. J. Pharmacol. Exp. Ther., 282: 1396-1401 (1997).

We claim:

1. A method of treating Parkinson's disease in a human that comprises administering a pharmaceutical composition comprising a pharmaceutically effective amount of a GDNF protein product and at least one pharmaceutically acceptable vehicle, excipient, or diluent to one or both putamen of a human in need thereof.

2. A method of treating Parkinson's disease in a human that comprises
assessing dopaminergic function in one or both putamen of said human, pre-operatively;
identifying at least one site of dopaminergic dysfunction within one or both putamen;
administering a pharmaceutical composition comprising a pharmaceutically effective amount of a GDNF protein product and at least one pharmaceutically acceptable vehicle, excipient, or diluent to one or more of said sites; and, optionally,
assessing dopaminergic function at one or more of said sites post-operatively, at least once.

3. A method of increasing the function of dopaminergic neurons in a human that comprises administering a pharmaceutical composition comprising a pharmaceutically effective amount of a GDNF protein product and at least one pharmaceutically acceptable vehicle, excipient, or diluent to one or both putamen of a human in need thereof.

4. A method of increasing the uptake of dopamine by dopaminergic neurons in a human that comprises administering a pharmaceutical composition comprising a pharmaceutically effective amount of a GDNF protein product and at least one pharmaceutically acceptable vehicle, excipient, or diluent to one or both putamen of a human in need thereof.

5. A method of regenerating dopaminergic neurons in a human that comprises administering a pharmaceutical composition comprising a pharmaceutically effective amount of a GDNF protein product and at least one pharmaceutically acceptable vehicle, excipient, or diluent to one or both putamen of a human in need thereof.

6. A method of protecting dopaminergic neurons susceptible to degeneration in a human that comprises administering a pharmaceutical composition comprising a pharmaceutically effective amount of a GDNF protein product and at least one pharmaceutically acceptable vehicle, excipient, or diluent to one or both putamen of a human in need thereof.

7. A method of treating Parkinson's disease in a human that comprises administering a pharmaceutical composition comprising a pharmaceutically effective amount of a GDNF protein product and at least one pharmaceutically acceptable vehicle, excipient, or diluent to the central region of at least one putamen of a human in need thereof.

8. A method of treating Parkinson's disease in a human that comprises administering a pharmaceutical composition comprising a pharmaceutically effective amount of a GDNF protein product and at least one pharmaceutically acceptable vehicle, excipient, or diluent to the postero-dorsal region of at least one putamen of a human in need thereof.

9. The method of any one of claims 1 through 8, wherein said vehicle, excipient, or diluent comprises sodium chloride and sodium citrate.

10. The method any one of claims 1 through 9, wherein said GDNF protein product is r-metHuGDNF.

11. The method any one of claims 1 through 10, wherein assessing dopaminergic function comprises assessing dopamine uptake or dopamine storage.

12. The method of any one of claims 2, 9, or 10, wherein said site of dopaminergic dysfunction is the postero-dorsal region of one or both putamen.

13. The method of any one of claims 2, 9, or 10, wherein said site of dopaminergic dysfunction is the central region of one or both putamen.

14. A kit comprising:
(a) one or more supplies of a pharmaceutical composition comprising a GDNF protein product and a pharmaceutically acceptable vehicle, excipient, or diluent; and
(b) one or more provisions for refilling an implanted drug delivery device with said pharmaceutical composition.

15. The kit of claim 14, wherein the GDNF protein product is r-metHuGDNF.

16. The kit of claim 14, wherein said vehicle, excipient, or diluent comprises sodium chloride and sodium citrate.

17. A kit comprising:
(a) one or more supplies of a pharmaceutical composition comprising a GDNF protein product and a pharmaceutically acceptable vehicle, excipient, or diluent; and
(b) one or more provisions for refilling an implanted drug delivery device with said pharmaceutical composition; and
(c) optionally, instructions for refilling said drug delivery device.

18. The kit of claim 17, wherein the GDNF protein product is r-metHuGDNF.

19. The kit of claim 18, wherein said vehicle, excipient, or diluent comprises comprises an aqueous buffer.

20. The kit of claim 19, wherein said aqueous buffer comprises sodium chloride and sodium citrate.

21. The kit of claim 20, wherein said provision for refilling said drug delivery device is one or more syringes.

22. The kit of claim 21, wherein said provision for refilling said drug delivery device is one or more supplies of a pharmaceutically acceptable diluent.

23. The kit of claim 22, wherein said diluent is citrate buffered saline, pH of about 4.5 to about 5.5.

24. The kit of claim 23, wherein the citrate buffered saline comprises 150 mM sodium chloride and 10 mM sodium citrate, pH 5.0.

25. The kit of claim 24, wherein the pharmaceutical composition, the diluent, and the syringes are sterile.
